(19) United States
(12) Patent Application Publication
(10) Pub. No.: US 2026/0306853 A1
(43) Pub. Date: Oct. 8, 2026

(54) DETECTION AND VERIFICATION METHODS AND SYSTEMS FOR LEFT BUNDLE BRANCH PACING CAPTURE

(71) Applicants: Michael V Orlov, Newton, MA (US); Zachary Whinnett, Rickmansworth Chorleywood (GB)

(72) Inventors: Michael V Orlov, Newton, MA (US); Zachary Whinnett, Rickmansworth Chorleywood (GB)

(21) Appl. No.: 19/460,062

(22) Filed: Jan. 26, 2026

Related U.S. Application Data
(60) Provisional application No. 63/749,268, filed on Jan. 24, 2025.

(51) Int. Cl. A61N 1/37; A61N 1/02; A61N 1/368

(52) U.S. Cl. CPC A61N 1/3712; A61N 1/025; A61N 1/36842

(57) ABSTRACT

Methods and systems for detecting and verifying left bundle branch pacing capture are disclosed. The invention provides objective, quantifiable criteria for differentiating left bundle branch capture from left ventricular septal capture by measuring the direction of electrical impulse propagation and time delays along the lateral wall of the left ventricle. A novel bidirectional pacing protocol measures conduction times in both anterograde and retrograde directions, exploiting the preferential anterograde conduction properties of the specialized His-Purkinje system. The resulting bidirectional conduction time differential (delta) provides a functional assessment of conduction system engagement, with a significant delta (20 milliseconds or greater) confirming left bundle branch capture. The invention enables real-time verification using noninvasive electrocardiographic imaging during septal electrode implantation, providing immediate visual feedback of ventricular activation patterns and enabling iterative optimization of lead position. Chronic monitoring methods detect late loss of left bundle branch capture with automated alerting and corrective actions.

### CROSS-REFERENCE DATA

This patent application claims a priority date benefit from a co-pending U.S. Provisional Patent Application No. 63/749,268 filed on 24 Jan. 2025 and entitled “DETECTION AND VERIFICATION METHODS FOR LEFT BUNDLE BRANCH PACING CAPTURE,” incorporated herein by reference in its entirety.

### BACKGROUND

Without limiting the scope of the invention, its background is described in connection with cardiac pacing. More particularly, the invention describes methods and systems for detecting proper pacing capture of a left bundle branch of a heart. The invention also describes methods of differentiating between a capture of the interventricular septum and a capture of the left bundle branch of the cardiac conduction system when delivering pacing therapy by a septal electrode. Additionally, the invention encompasses novel bidirectional pacing protocols and noninvasive electrocardiographic mapping techniques for verifying left bundle branch capture during electrode implantation and chronic monitoring.

### Overview of Normal Cardiac Conduction

The electrical event, the wave of depolarization, is the trigger for myocardial contraction. In a normal heart, propagation of the electrical impulse during the systolic activation of the heart progresses from a sinoatrial (SA) node, across the atria, to the atrioventricular node. At that point, there is usually a delay of approximately 100-200 milliseconds that allows the atria to complete pumping blood before the impulse is transmitted through the atrioventricular bundle. Following the delay, the impulse travels through the atrioventricular bundle and bundle branches (including a left bundle branch) located in the interventricular septum in a direction from the base of the heart toward the apex of the heart. After reaching the apex of the heart, the impulse propagation changes direction and travels through the Purkinje fibers of the lateral wall in a direction from apex to base, causing a lateral wall contraction.

The left ventricle and the entire heart contract in a certain organized manner, including a synchronous contraction of the interventricular wall and an almost simultaneous contraction of the lateral wall, sometimes with a small delay. This physiological activation pattern is characterized by rapid conduction through the specialized His-Purkinje conduction system, with conduction velocities of 2-3 meters per second in Purkinje fibers compared to only 0.3-0.4 meters per second in ordinary ventricular myocardium. The specialized conduction system ensures that the entire left ventricle is activated within approximately 60-80 milliseconds, resulting in efficient and synchronous ventricular contraction.

### Abnormalities of Cardiac Conduction and Pacing Therapy

A variety of abnormalities and disruptions of normal myocardial contraction may be encountered and treated by an electrophysiologist. In some patients, there are problems transmitting the electrical impulse through the atrioventricular bundle and further through the bundle branches of the cardiac conduction system. In other patients, there are other causes of arrhythmia and improper or dyssynchronous heart contractions. A number of these ailments may be treated by pacing therapy. One type of pacing therapy involves implanting a pacing electrode into an interventricular septum.

### Clinical Applications of Septal Electrodes

In broad terms, the interventricular septum is an important site for treating various arrhythmias. A septal catheter or an electrode placed in this region can be used to treat several types of cardiac arrhythmias. One significant category of arrhythmias treatable with septal electrodes is ventricular tachycardia (VT) originating from a septal substrate. These VTs often arise from areas of myocardial scarring, particularly in patients with ischemic or non-ischemic cardiomyopathies. Septal VTs may involve critical components of the re-entry circuit confined to the endocardium, the Purkinje system, or intramural areas. The septal catheter/electrode can be used for mapping the substrate, identifying areas of abnormal potentials, and subsequently for ablation procedures.

Furthermore, septal catheters/electrodes can be valuable in treating ventricular arrhythmias involving the specialized conduction system, such as bundle branch re-entrant tachycardias or fascicular VTs. These arrhythmias often involve the septal regions where the conduction system resides, and precise mapping and ablation using septal catheters/electrodes can be crucial for successful treatment.

### Conduction System Pacing: an Emerging Therapy

Septal electrodes are also used as a promising alternative to traditional right ventricular and biventricular pacing for avoiding or correcting heart failure associated with ventricular dyssynchrony, which is an inefficient, asynchronous contraction associated with left bundle branch block (LBBB) or similar conditions. This approach, known as conduction system pacing (CSP), aims to maintain or restore cardiac synchrony and physiological conduction patterns by directly capturing the left bundle branch or its surrounding area. Two main techniques utilizing septal electrodes are His-bundle pacing and left bundle branch area pacing (LBBAP). These methods offer potential advantages such as more natural ventricular activation, improved response rates, and reduced lead-related complications such as absence of diaphragmatic stimulation and others, compared to conventional cardiac resynchronization therapy.

Prospective studies have demonstrated that LBBAP significantly reduces composite clinical endpoints compared to traditional right ventricular pacing. In published studies, at 2-year follow-up, patients receiving LBBAP showed a 22% reduction in the composite endpoint of death, heart failure hospitalization, or need for cardiac resynchronization therapy upgrade. The benefit was particularly pronounced in patients with high pacing burden (>20% ventricular pacing), where LBBAP reduced the event rate from 26.1% to 8.4%. The global cardiac implantable electronic devices market reflects the substantial commercial and clinical importance of optimizing pacing therapy.

### The Challenge of Verifying Left Bundle Branch Capture

Direct left bundle branch (LBB) capture is the goal of CSP as it provides the most physiologic pattern of cardiac activation. However, septal capture may be present instead if the lead tip did not advance into the vicinity of the left bundle branch and its tributaries. Depending on the depth of septal penetration, right ventricular septal, deep septal, and left ventricular septal pacing (LVSP) may occur. Importantly, septal pacing and even LVSP, which is anatomically the closest to the LBB location, do not provide the same degree of synchrony and restoration of normal physiologic conduction and, even while appearing very similar on ECG, may result in continued dyssynchrony and heart failure.

### Anatomical and Electrophysiological Challenges

Differentiating LBB capture from LVSP during septal electrode implantation presents significant challenges due to the complex anatomy and electrophysiology of the interventricular septum. The highly-variable structure of the LBB, lying beneath the left ventricular septal endocardium with extensive interfascicular connections, makes precise targeting difficult without also capturing the surrounding septal myocardium. This anatomical proximity often results in fused (nonselective) QRS complexes.

Electrophysiological challenges include similar QRS morphologies on the surface ECG for both LBB and LVS capture, typically showing a right bundle branch block pattern in lead V1. Some known diagnostic techniques include observing output-dependent QRS transitions, identifying left bundle branch potentials on intracardiac electrograms, measuring stimulus to left ventricular activation time on the standard ECG, and using programmed ventricular stimulation. However, these methods have significant limitations and may not be universally applicable.

### Limitations of Current Diagnostic Methods

ECG-Based Criteria. Studies have shown that metrics differentiating LBBAP and LVSP frequently overlap, complicating diagnostic accuracy. The V6 R-wave peak time (V6-RWPT), commonly used as a diagnostic criterion, has overlapping values between LBB capture and septal capture, with only 60-90% of patients demonstrating clear LBB capture by all available criteria. This overlap creates diagnostic uncertainty during procedures. The presence of narrow QRS duration (<130 ms) on paced ECG, often cited as evidence of successful LBB capture, can also occur with pure septal capture that does not engage the left bundle branch proper. Furthermore, a change of QRS morphology during output reduction, while suggestive of selective capture, is not a reliable diagnostic indicator in all patient populations.

Intracardiac Electrograms. Recording of left bundle branch potentials provides strong evidence of LBB proximity, but such potentials are not recordable in all patients. Recent studies indicate that LBB potentials are identified in only 50-70% of successful LBB captures, suggesting that their absence does not definitively rule out conduction system engagement. Additionally, the presence of LBB potentials does not guarantee that pacing will selectively capture the conduction system rather than surrounding myocardium. The interpretation of electrograms may also be subjective and variable between operators, reducing standardization of diagnostic criteria.

Output-Dependent Transitions. Some patients demonstrate a transition in QRS morphology when pacing output is reduced, theoretically indicating a shift from nonselective capture (LBB plus surrounding myocardium) to selective LBB capture. However, this phenomenon is not universally present in successfully captured LBB pacing, occurring in only approximately 40-50% of cases. Similar transitions can occur with deep septal capture that does not engage the LBB, making this criterion insufficient for differentiation. Furthermore, variability in the relationship between pacing output and QRS morphology between patients and between different septal locations limits the reliability of this approach.

Programmed Stimulation. Techniques such as evaluating for retrograde His activation during ventricular pacing or assessing conduction delays during extrastimulus testing provide functional evidence of conduction system engagement but require additional time, expertise, and interpretation, and are not standardized across institutions. These methods necessitate specialized equipment and training, limiting their applicability to centers with advanced electrophysiology capabilities. The complexity of interpretation and high technical demands make these methods impractical for routine use during device implantation procedures.

Clinical data indicate that even among experienced operators, 1.3% to 4% of patients experience loss of LBB capture during short-term follow-up, and distinguishing between true LBB capture and deep septal capture remains challenging in a substantial proportion of cases. This diagnostic uncertainty has significant clinical consequences, as patients receiving septal myocardial pacing versus true LBB capture demonstrate different long-term clinical outcomes, with true LBB capture associated with superior improvements in left ventricular ejection fraction, reduction in QRS duration, and symptom relief.

### Physiological Basis for the Present Invention

### Activation Direction and Timing as Diagnostic Markers

The present invention is based on the recognition that left bundle branch capture produces characteristic patterns of electrical impulse propagation along the lateral wall of the left ventricle that differ fundamentally from septal myocardial capture. In patients with normal left bundle branch conduction, the left ventricle (and, in particular, its lateral wall) activates in a direction from apex to base, reflecting the rapid transmission of electrical impulses through the Purkinje network to the apex (FIG. 1), followed by endocardial-to-epicardial activation proceeding toward the base. The time delay from septal activation to lateral wall activation is typically 60-80 milliseconds, representing rapid specialized conduction. This apex-to-base activation pattern results from the normal physiology wherein impulses traveling through the left bundle branch reach the apical regions first, with activation then propagating toward the base.

When the left bundle branch conductivity is impaired, as in left bundle branch block or right ventricular pacing-induced dyssynchrony, the activation of the left ventricle and lateral wall progresses in an abnormal pattern, which may include base-to-apex direction, side-to-side patterns, or other non-physiological sequences. The time delay from septal activation to lateral wall activation is markedly prolonged, often exceeding 90-100 milliseconds, reflecting slow myocardial conduction velocities at 0.3-0.4 m/s rather than the rapid 2-3 m/s conduction through Purkinje fibers. The loss of the normal apex-to-base activation gradient is a hallmark of conduction system dysfunction.

When septal pacing captures the left bundle branch, the physiological activation pattern is restored: apex-to-base propagation along the lateral wall with shortened conduction delays similar to normal sinus rhythm, typically 40-80 milliseconds. This restoration of normal activation patterns, documented both on intracardiac recordings and noninvasive ECG assessment, confirms engagement of the specialized conduction system. In contrast, when septal pacing captures only the ventricular myocardium without engaging the specialized conduction system, the abnormal activation patterns persist or worsen, and prolonged conduction delays continue or increase. The absence of restoration of normal apex-to-base gradients and persistence of elevated conduction delays indicate failure to achieve LBB capture.

### Bidirectional Conduction Properties of Specialized Tissue

A further physiological basis for the present invention relates to the directional conduction properties of the specialized cardiac conduction system. The His-Purkinje system exhibits preferential anterograde (normal physiological direction) conduction, with conduction velocities of 2-3 meters per second when impulses propagate from the His bundle through the bundle branches and Purkinje fibers to the ventricular myocardium. However, retrograde conduction, such as from ventricular myocardium back into the proximal specialized conduction system, faces significant impedance and functional barriers.

This asymmetry arises from several factors: (1) the Purkinje-myocardial junction exhibits directional differences in conduction delay, with retrograde transmission being impeded by refractory properties and decremental conduction; (2) the specialized conduction tissue typically exhibits unidirectional block or preferential conduction in the physiological direction due to the directional properties of gap junctions and cellular arrangement; and (3) the large electrical load presented by ventricular myocardium when conducting retrogradely into the narrow specialized fibers creates source-sink mismatch that prevents effective retrograde propagation into the conduction system.

The present invention uses these physiological properties by introducing bidirectional pacing protocols. When pacing from a septal electrode that has successfully captured the left bundle branch, electrical impulses conduct rapidly in the anterograde direction through the specialized conduction system to the lateral wall (short conduction time, e.g., 40-60 milliseconds). This rapid conduction reflects the high velocity of impulse propagation through Purkinje fibers (2-3 m/s) and the direct engagement of the fast-conducting specialized tissue.

When pacing in the reverse direction from lateral wall electrodes, the impulse must propagate slowly through ventricular myocardium (conduction velocity 0.3-0.4 m/s) and cannot effectively enter the specialized conduction system in the retrograde direction due to the unidirectional properties and impedance of the Purkinje-myocardial junction. This results in much longer conduction times (e.g., 80-100 milliseconds or greater) to reach the septal electrode. The difference between these conduction times (also referred to as the bidirectional conduction time differential or “delta”) provides a functional assessment of conduction system engagement. A large delta (typically >20-30 milliseconds) indicates that the forward pacing direction utilizes fast specialized tissue (short delay) while the reverse direction must use slow myocardial tissue (long delay), confirming LBB capture.

In contrast, when the septal electrode captures only ventricular myocardium without engaging the left bundle branch, conduction is slow in both directions (both via myocardial pathways at 0.3-0.4 m/s), resulting in long conduction times bidirectionally (80-100+ ms in both directions) and minimal delta (typically <10-20 milliseconds) between forward and reverse pacing. This bidirectional conduction time differential measurement thus provides a novel, physiologically-based diagnostic criterion for differentiating LBB capture from septal myocardial capture.

The bidirectional delta measurement has significant advantages over existing methods: (1) it is objective and quantifiable, not requiring subjective interpretation of ECG morphology; (2) it directly assesses functional engagement of the conduction system rather than indirect morphologic markers; (3) it does not require the presence of recordable LBB potentials; (4) it is applicable to all patients regardless of baseline QRS morphology or presence of pre-existing conduction abnormalities; and (5) it can be performed using standard pacemaker equipment capable of pacing from multiple electrodes, enabling widespread clinical adoption without requiring specialized equipment beyond current standards.

### Emerging Noninvasive Mapping Technologies

### Electrocardiographic Imaging (ECGi)

Electrocardiographic imaging represents a significant advancement in noninvasive cardiac electrophysiology. ECGi uses a large number of body surface electrodes (typically, at least 64, but the number of electrodes may be higher, as many as 120-256 electrodes, or lower, such as when used for a specific purpose, for example, assessing pacing capture type) combined with three-dimensional cardiac geometry obtained from computed tomography or magnetic resonance imaging to reconstruct epicardial and endocardial electrical activation patterns through inverse solution of the electrocardiographic problem. This technology has been validated in numerous studies and can localize arrhythmic foci with an accuracy better than 10 millimeters compared to invasive mapping. The ECGi system provides isochrone maps displaying the temporal sequence of ventricular activation, allowing visualization of conduction pathways and identification of sites of earliest and latest activation.

Recent technological developments have enabled real-time or near-real-time ECGi during cardiac procedures, including septal electrode implantation. “Imageless” ECGi systems using artificial intelligence-derived cardiac geometric models eliminate the need for pre-procedural computed tomography or magnetic resonance imaging, enabling immediate deployment in the electrophysiology laboratory. Such systems can provide visual feedback to the implanting physician within 2-5 seconds of each test pacing maneuver, allowing iterative optimization of electrode depth and position based on observed activation patterns. These advances have dramatically improved the practical utility of ECGi for real-time procedural guidance.

ECGi has been evaluated specifically for guiding left bundle branch pacing and assessing electrical resynchronization. Studies demonstrate that ECGi can detect the transition from septal myocardial pacing (characterized by delayed lateral wall activation occurring at 90-120 milliseconds and abnormal activation sequences) to left bundle branch pacing (characterized by early septal activation followed by rapid, synchronous lateral wall activation with physiological apex-to-base patterns and total left ventricular activation time of 60-80 milliseconds). In clinical studies, 91.6% of patients achieving electrical resynchronization by ECGi criteria showed successful LBB capture, compared to only 60-70% when using standard ECG criteria alone. This substantially improved diagnostic accuracy demonstrates the value of ECGi for confirming conduction system engagement.

The quantitative parameters measurable by ECGi include: (1) total left ventricular activation time (typically <80 ms for LBB capture vs. >90 ms for septal capture); (2) activation time from earliest to latest sites; (3) spatial distribution of activation; (4) direction of propagation along the lateral wall (apex-to-base vs. other patterns); and (5) presence of early septal activation indicating pacing site capture. These objective, quantifiable measurements eliminate subjective interpretation and provide physiologically meaningful data regarding the quality of cardiac synchrony achieved by the pacing strategy.

### Body Surface Potential Mapping

Body surface potential mapping (BSPM) represents an intermediate approach between standard 12-lead electrocardiography and full ECGi reconstruction. BSPM employs dense arrays of 64-252 electrodes to record spatial and temporal distribution of cardiac electrical activity on the torso surface. While BSPM does not reconstruct detailed three-dimensional epicardial activation maps, it can detect regional timing differences and activation sequence changes that correlate with left bundle branch capture versus septal capture. BSPM offers advantages of lower computational complexity and cost compared to full ECGi systems while providing substantially more spatial resolution than conventional 12-lead ECG.

The technical basis for BSPM relates to the principle that body surface potentials, when recorded from many sites simultaneously, contain information about the underlying cardiac electrical activity. By analyzing the distribution of potentials across the chest, information can be derived about the timing of activation at different sites and the direction of electrical propagation. Body surface potential maps display this spatial distribution, with different colors representing different potential values at different time points during the cardiac cycle. The evolution of these maps during successive time intervals reveals the sequence of ventricular activation.

Clinical studies have demonstrated that BSPM can optimize cardiac resynchronization therapy by identifying the site of latest left ventricular activation and confirming electrical synchrony after intervention. The application of BSPM to conduction system pacing verification represents a natural extension of this technology. BSPM can be used to identify the transition from abnormal activation patterns (in patients with baseline LBBB or RV pacing-induced dyssynchrony) to normalized apex-to-base activation patterns (indicating LBB capture), and to detect the prolonged delays characteristic of septal myocardial capture.

### High-Resolution Multi-Lead Electrocardiography

Advanced multi-lead electrocardiographic systems recording from 18 to 64 leads simultaneously at high sweep speeds (100-250 mm/s) represent a practical intermediate approach between standard 12-lead ECG and full ECGi. These systems can detect small timing differences between different regions of the left ventricle by analyzing the relationship between pacing stimulus and QRS onset at different leads, and by detecting subtle morphologic changes reflective of different activation sequences. While less sophisticated than full ECGi, high-resolution multi-lead ECG can be deployed in standard electrophysiology laboratories without requiring CT or MRI imaging or extensive computational analysis.

### Other ECG-based Methods

Other derivatives of standard 12-lead+ECG and vectorcardiography have been used to describe the timing and direction of myocardial activation. These metrics can also be used to noninvasively measure the delay to the lateral wall activation when attempting to differentiate between various types of conduction system capture.

### Clinical Significance and Patient Outcomes

The clinical importance of accurately differentiating left bundle branch capture from septal myocardial capture cannot be overstated. Patients who receive true LBB pacing demonstrate superior clinical outcomes compared to those with septal myocardial pacing, even when conventional ECG criteria suggest adequate resynchronization. In comparative studies, LBB capture is associated with greater reductions in QRS duration (34% reduction vs. 11% with conventional pacing), improved left ventricular ejection fraction (from 35% to 48% vs. smaller improvements with septal pacing), reduced heart failure hospitalizations, and lower all-cause mortality.

The quality of life improvements experienced by patients receiving successful LBB pacing are also substantial. Patients demonstrate functional improvement in New York Heart Association symptom classification, improved exercise tolerance, reduced dyspnea, and improved overall quality of life measures. These clinical benefits justify the effort required to achieve and verify true LBB capture during implantation procedures.

The procedural challenges of achieving and confirming LBB capture create practical barriers to the widespread adoption of conduction system pacing. Operators often face uncertainty during procedures when available diagnostic criteria provide conflicting results. This uncertainty may lead to: (1) abandonment of conduction system pacing in favor of conventional biventricular pacing despite potential advantages; (2) acceptance of suboptimal septal myocardial capture mistakenly believed to be LBB capture; or (3) prolonged procedure times attempting to optimize lead position based on ambiguous criteria. Such prolonged procedures increase patient discomfort, radiation exposure (when fluoroscopy is used), contrast exposure (during venography), and procedural costs while potentially delaying achievement of optimal therapy.

Furthermore, chronic monitoring for loss of LBB capture after successful implantation is not well-established in current practice. Lead migration, fibrosis around the lead, or changes in capture threshold over time may result in late loss of conduction system engagement, converting effective LBB pacing to ineffective septal myocardial pacing. Without reliable methods for detecting such loss, patients may experience gradual deterioration in clinical status that is not recognized as being due to pacing failure. Such late loss of capture may lead to delayed recognition of device dysfunction, worsening heart failure, hospitalizations, and unnecessary prolongation of inadequate pacing therapy.

The global shift toward conduction system pacing as the preferred modality for cardiac resynchronization therapy underscores the urgency of developing robust, standardized verification methods. Recent clinical consensus statements suggest that conduction system pacing has the potential to replace conventional biventricular pacing as the standard of care for patients with heart failure and dyssynchrony. Leading cardiac societies are incorporating LBBAP into guidelines as a preferred approach. Realizing this potential requires addressing the current limitations in confirming LBB capture and enabling reliable identification of successful procedures.

### Summary of the Need

The need exists, therefore, for novel systems and methods for better identification of LBB capture and more reliable differentiation between LBB and LVS capture when implanting a septal electrode for conduction system pacing and other purposes. Specifically, there is a need for:

Objective, quantifiable criteria for differentiating LBB capture from septal myocardial capture that do not rely solely on subjective interpretation of QRS morphology or require the presence of recordable LBB potentials that may not be present in all patients. Reference is made here to FIG. 3, which shows LBB potential (white arrows) at the location with LBB pacing capture, and no LBB potential at the left septal site without LBB capture left panel. FIG. 2 shows the corresponding paced ECG, and FIG. 4 shows the corresponding lateral wall measurements. Such criteria should provide specific numerical thresholds (e.g., activation timing less than a specified value indicating LBB capture, or greater than another value indicating septal capture) that enable consistent application across different operators and institutions.

Functional assessment methods that evaluate the physiological consequences of pacing (activation patterns, conduction velocities, synchrony) rather than indirect markers, providing more direct evidence of conduction system engagement. These methods should measure actual electrical conduction properties rather than inferring them from QRS morphology or other indirect indicators.

Bidirectional pacing protocols that exploit the inherent directional properties of specialized conduction tissue to reveal conduction system capture through differential conduction time measurements not achievable with unidirectional pacing alone. Such protocols should provide quantifiable measurements (delta values) that clearly differentiate successful conduction system engagement from myocardial capture.

Noninvasive verification techniques using advanced electrocardiographic mapping technologies (ECGi, BSPM, high-resolution multi-lead ECG and others) that can provide real-time procedural guidance and enable chronic monitoring without requiring permanently implanted sensing/pacing electrodes. These noninvasive approaches should be practical for use in standard electrophysiology laboratories and should not require extensive pre-procedural preparation or post-procedural analysis.

Standardized Methods Applicable Across Diverse Patient Populations, Including those with pre-existing conduction abnormalities, myocardial scarring, or other complexities that make conventional diagnostic criteria unreliable. Methods should be equally effective in patients with or without baseline LBBB, in those with prior myocardial infarction or cardiomyopathy, and across the full spectrum of left ventricular ejection fractions encountered in clinical practice.

Chronic monitoring capabilities for detecting late loss of LBB capture due to lead migration, fibrosis, or threshold changes, enabling timely intervention before clinical deterioration occurs. Such monitoring should be feasible with implanted devices during routine follow-up and should alert clinicians to pacing failure so that corrective action can be taken.

Practical implementation that integrates with existing pacemakers and implantable cardioverter-defibrillator devices without requiring new hardware development, enabling rapid clinical adoption and widespread availability to patients who would benefit from conduction system pacing.

The present invention addresses these needs by providing comprehensive methods and systems for detecting and verifying left bundle branch capture using activation direction and timing assessment, novel bidirectional pacing protocols, and noninvasive electrocardiographic imaging technologies. These approaches offer objective, physiologically-based criteria for confirming conduction system engagement during initial implantation and throughout chronic follow-up, thereby optimizing patient outcomes and facilitating the broader adoption of this promising pacing modality. By enabling reliable verification of true left bundle branch capture, the invention removes a major barrier to the widespread clinical implementation of conduction system pacing, supporting the transition to physiologic pacing as the new standard of care for cardiac resynchronization therapy and bradycardia pacing indications.

### SUMMARY

Accordingly, it is an object of the present invention to overcome these and other drawbacks of the prior art and to establish reliable, quantifiable methods for identifying successful left bundle branch capture during septal electrode implantation and chronic monitoring. Rather than relying upon subjective electrocardiographic interpretation or recordable potentials that may not be present in all patients, the invention provides objective measurements of electrical impulse propagation direction (apex-to-base versus abnormal patterns) and conduction timing along the lateral wall of the left ventricle, with specific quantitative thresholds enabling consistent identification across diverse patient populations.

It is another object of the present invention to develop bidirectional pacing methodologies that differentiate left bundle branch capture from septal myocardial capture by measuring conduction times in both anterograde and retrograde directions. The specialized conduction system exhibits preferential anterograde conduction (2-3 m/s through Purkinje fibers) while retrograde conduction faces significant impedance. By measuring the bidirectional conduction time differential (delta), the invention provides a functional measure of conduction system engagement, with a substantial delta (20-30 milliseconds or greater) confirming left bundle branch capture and a minimal delta (<10-20 milliseconds) indicating septal myocardial capture.

It is a further object of the present invention to translate emerging electrocardiographic imaging technologies (specifically, electrocardiographic imaging (ECGi), body surface potential mapping (BSPM), high-resolution multi-lead electrocardiography, vectorcardiography imaging) and extended applications of 12-lead plus ECG into practical tools for procedural guidance and chronic monitoring. The invention enables real-time activation mapping during septal electrode implantation with immediate visual feedback, allowing precise optimization of lead position and confirmation of successful left bundle branch capture. Additionally, the invention establishes comprehensive chronic monitoring methods for detecting late loss of left bundle branch capture and enabling timely clinical intervention through automated alerting and corrective actions.

The present invention describes a method for detecting left bundle branch capture using activation direction and timing measurements. The method comprises deploying a septal electrode, monitoring the direction of electrical impulse propagation along the lateral wall and measuring time delays from septal to lateral wall activation, initiating pacing, and identifying left bundle branch capture upon restoration of apex-to-base propagation or shortening of delays by at least 15% compared to baseline; or upon maintaining this physiological activation pattern in patents with intrinsic conduction via the left conduction system. The invention further describes measurement methodologies using at least two lateral wall electrodes (optionally including quadripolar coronary sinus leads or surface ECG electrodes), define multiple options for measuring time delays (pacing stimulus to R-wave peak, to earliest/peak activation at lateral wall electrodes, or QRS onset to earliest activation), and establish quantitative thresholds for diagnosis (left bundle branch capture: <80 milliseconds delay; left ventricular septal capture: >85-95 milliseconds delay).

In other embodiments, the present invention introduces a novel bidirectional pacing method for differentiating left bundle branch capture from septal myocardial capture. The method comprises deploying septal and lateral wall electrodes, initiating a first pacing protocol from the septal electrode and measuring conduction time to the lateral wall electrodes, initiating a second pacing protocol from the lateral wall electrodes and measuring conduction time to the septal electrode, calculating the bidirectional conduction time differential (delta), and identifying left bundle branch capture when delta exceeds a threshold value (10-50 milliseconds, preferably 20-30 milliseconds). The invention describes detailed implementations using quadripolar electrode leads, dynamic adjustment of thresholds based on patient-specific factors (baseline QRS duration, presence of left bundle branch block, myocardial scarring, left ventricular ejection fraction), validation across multiple pacing output voltages, clinical application for therapy delivery decisions (left bundle branch capture confirmed: continue septal pacing; capture not confirmed: switch to biventricular pacing), chronic monitoring for detection of late loss of capture with automated corrective actions, and apparatus/system implementation combining pacing modules, sensing modules, processing modules calculating conduction times and delta values, and output modules indicating capture status.

Furthermore, the present invention describes methods for real-time verification of left bundle branch capture during septal electrode implantation using electrocardiographic imaging (ECGi) and related noninvasive technologies. These methods comprise deploying an ECGi system with body surface electrodes, acquiring three-dimensional cardiac geometry from imaging or artificial intelligence-derived models, continuously recording electrical signals during implantation, generating real-time activation maps as the septal electrode is advanced into the interventricular septum at multiple depths, comparing activation maps to identify transitions from left ventricular septal pacing patterns (delayed lateral wall activation, abnormal sequences) to left bundle branch pacing patterns (early septal activation, rapid synchronous lateral wall activation, apex-to-base direction, shortened left ventricular activation time), and confirming successful left bundle branch capture when activation maps demonstrate the left bundle branch pacing pattern with left ventricular activation time shortened by at least 15% compared to baseline. Also described are ECGi system components (64-256 body surface electrodes, computational processing for inverse electrocardiography reconstruction, measurement of left ventricular activation time), imageless ECGi implementation using artificial intelligence-derived cardiac geometric models enabling real-time feedback within 2-5 seconds of each test pacing maneuver, advanced ECGi capabilities (quantitative electrical resynchronization measurement, visualization of latest left ventricular activation sites, assessment of pacing site proximity to left bundle branch, detection of anodal capture, generation of resynchronization assessment reports), and alternative noninvasive measurement modalities including body surface potential mapping, high-resolution multi-lead electrocardiography with 18-64 leads, and others.

Overall, the present invention describes methods and systems for detecting left bundle branch capture through complementary approaches: objective physiologic criteria based on activation patterns and timing, novel bidirectional pacing protocols exploiting conduction system properties, and real-time noninvasive electrocardiographic imaging guidance.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a diagram of a heart and key locations of an electrical conduction system,

FIGS. 2 and 3 show exemplary ECG tracings and intracardiac electrograms for different pacing depths and capture types,

FIG. 4 is a table showing various delays as recorded using a quadripolar lead,

FIG. 5 is a graph showing average delays for different pacing scenarios recorded in a group of 17 patients,

FIG. 6 is an exemplary radiographic image (Example 1) of a heart showing a placement of various electrodes and leads,

FIG. 7 is an electrocardiogram tracing for Example 1 showing baseline conduction,

FIG. 8 is another electrocardiogram tracing for Example 1 under Left Bundle Branch pacing for electrode 1,

FIG. 9 is the same as in FIG. 4, but for electrode 2,

FIG. 10 is an electrocardiogram tracing for Example 1 showing baseline conduction as seen by electrode 3,

FIG. 11 is the same as in FIG. 6, but under a condition of left bundle branch pacing,

FIG. 12 is an electrocardiogram tracing for Example 1 showing baseline conduction as seen by electrode 4,

FIG. 13 is the same as in FIG. 8, but under a condition of left bundle branch pacing,

FIG. 14 is a radiographic image of another patient presenting with an RV leadless pacemaker, dyssynchrony, and heart failure (Example 2),

FIG. 15 is a table showing various delays as recorded by the 4 electrodes of a quadripolar coronary sinus lead under different pacing conditions (RV Micra pacing and LV septal pacing at high and low output), (Example 2), and

FIG. 16 is an ECG tracing for this patient (Example 2).

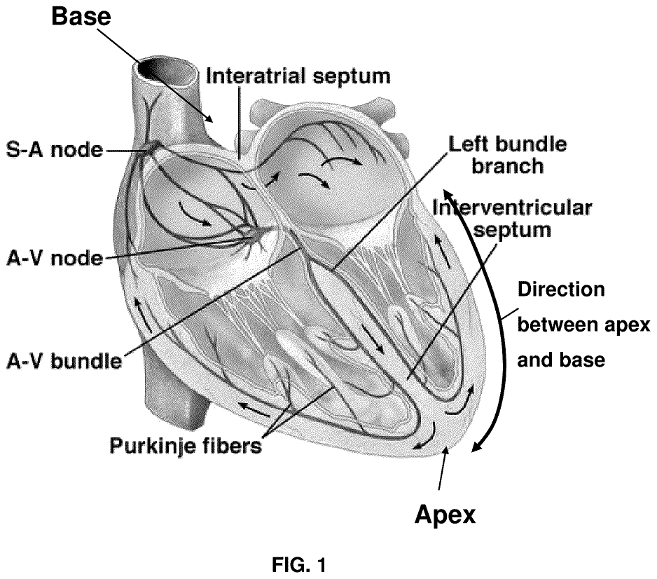

### DETAILED DESCRIPTION OF PREFERRED EMBODIMENTS OF THE INVENTION

The following description sets forth various examples and embodiments along with specific details to provide a thorough understanding of the claimed subject matter. It will be understood by those skilled in the art that claimed subject matter may be practiced without one or more of the specific details disclosed herein. Further, in some circumstances, well-known methods, procedures, systems, components, and circuits have not been described in detail in order to avoid unnecessarily obscuring claimed subject matter. It will be readily understood that the aspects of the present disclosure can be arranged, substituted, combined, and designed in a wide variety of different configurations, all of which are explicitly contemplated and make part of this disclosure.

The present invention takes advantage of the discovery that the direction and timing of electrical impulse propagation along the lateral wall of the left ventricle provides a physiologically meaningful marker of left bundle branch capture that is distinct from left ventricular septal myocardial capture. In patients with normal left bundle branch conduction, the left ventricle, and in particular its lateral wall, activates in a direction from apex to base. When the left bundle branch conductivity is impaired, as in left bundle branch block or right ventricular apical pacing-induced dyssynchrony, the activation of the left ventricle and lateral wall progresses in a reversed, abnormal direction from base to apex or in other non-physiological patterns. Upon successful left bundle branch pacing from a septal electrode, the physiological apex-to-base activation pattern is restored. When septal pacing captures only myocardium without engaging the specialized conduction system, abnormal activation patterns persist. Additionally, the time delays from septal activation to lateral wall activation are prolonged in the absence of left bundle branch capture, typically exceeding 85-95 milliseconds, but are shortened upon successful capture to less than 70-80 milliseconds, reflecting the superior conduction velocities of 2-3 meters per second through Purkinje fibers compared to 0.3-0.4 meters per second through myocardial tissue.

### Assessment of Activation Direction and Timing

### General Principles

Assessment of the direction of electrical impulse propagation along the lateral wall may be performed in a variety of ways. The term “along” in describing the line of propagation of the electrical impulse denotes a line between the apex and the base of the heart, as illustrated in reference figures. The direction of electrical impulse propagation along the heart may be determined through multiple methodologies, each offering distinct practical advantages and complementary information regarding the electrophysiologic state of the ventricle.

### Multiple Electrode Configuration Approaches

The direction of impulse propagation is determined by providing at least two or more electrodes spaced apart along a general direction between apex and base, or along a long axis of the heart. The electrodes may be positioned in multiple configurations, offering flexibility in clinical implementation:

Coronary Sinus Electrode Placement. Two or more electrodes in a branch of a coronary sinus allow sensing of lateral wall activation as impulses propagate along the venous system adjacent to the left ventricular lateral wall. This approach offers several advantages: the coronary sinus provides a well-established vascular conduit with low morbidity; the venous system runs along the lateral wall at a consistent anatomic location; and lead placement can typically be accomplished using standard percutaneous venous access techniques with standard equipment. This is the preferred approach due to its reliability, accessibility, and safety profile.

Endocardial Electrode Configuration. Electrodes positioned inside the left ventricle in contact with the endocardial surface of the heart allow direct sensing of transmural electrical activity at multiple left ventricular locations. This approach provides high signal quality and precise timing information but requires transseptal or transapical access, which carries a higher procedural risk than coronary sinus access. This approach may be selected when coronary sinus anatomy is unfavorable or when additional diagnostic information from endocardial mapping is desired.

Epicardial Electrode Placement. Electrodes positioned on the epicardial surface of the heart via pericardial access provide direct assessment of epicardial activation patterns. This approach requires deliberate pericardial access, either percutaneously via a subxiphoid approach or surgically, and is typically reserved for situations in which coronary sinus and endocardial approaches are not feasible or when comprehensive epicardial mapping is required.

Intracorporeal Electrodes. Electrodes positioned intracorporeally, such as on the housing (can) of a pacemaker or other internal locations within the thorax, can provide non-invasive body-sensing information regarding cardiac electrical activity. The pacemaker can serve as a remote sensing electrode and can detect far-field signals; while less precise than directly implanted leads, pacemaker case sensing provides continuous monitoring capability without additional hardware.

Body Surface Electrodes. Body surface electrodes attached to the skin at multiple locations on the chest provide a completely noninvasive assessment. Standard multi-lead electrocardiography uses 12 (or more) pre-defined lead positions; advanced systems may employ 64-256 body surface electrodes distributed around the thorax. Body surface electrodes eliminate procedural morbidity but provide lower spatial resolution than intracardiac electrodes.

Combined Electrode Approaches. Combinations of any of the above approaches may be employed. For example, a coronary sinus lead may provide primary intracardiac sensing while body surface electrodes provide complementary noninvasive confirmation of the observed activation patterns. This multimodal approach provides cross-validation and increases diagnostic confidence.

### Specific Implementation: Coronary Sinus Lead With Multiple Electrodes

One useful and preferred approach employs two or more electrodes located along the lateral wall of the left ventricle via coronary sinus placement. By knowing the position of electrodes along the lateral wall, the time of arrival of the electrical impulse (or other characteristics of the electrical signal, such as peak amplitude or slope) is compared between the at least two or more electrodes to determine the sequence of propagation and therefore the direction of propagation of the electrical impulse along the lateral wall of the heart. Multiple sensing electrodes may be provided on the same lead or distributed across multiple leads.

The lead containing multiple sensing electrodes may be inserted using any suitable blood vessel running at least in part along the lateral wall of the left ventricle. Coronary veins are a preferred location for situating the sensing electrodes as they provide a relatively safe vascular access while allowing placement of electrodes adjacent to the lateral wall of the left ventricle. The specific coronary vein selected (such as the great cardiac vein, anterior interventricular vein, or marginal branches) depends on individual patient anatomy but generally should run from the base toward the apex or near-parallel to the interventricular septum, providing electrodes spanning the apex-to-base dimension.

In other embodiments, spaced-apart sensing electrodes may be implanted into the pericardial space adjacent to the epicardial surface of the lateral wall. A subxiphoid pericardial access may be utilized to position electrodes along the epicardial surface. In further embodiments, at least two intraventricular sensing electrodes may be used if placed along the internal portion of the lateral wall of the left ventricle using transseptal or other intracardiac access techniques.

### Multi-Lead Electrocardiographic Assessment

The direction of impulse propagation may be determined by comparing electrocardiographic signals recorded from a multi-electrode chest lead configuration, such as a 64-electrode electrocardiographic system used for electrocardiographic imaging with three-dimensional reconstruction of electrical impulse propagation. Different locations around the chest allow detection of small differences between electrical signals that are indicative of the direction of electrical impulse propagation along the lateral wall and the left ventricle in general. Advanced computational methods may reconstruct the three-dimensional sequence of activation to provide detailed maps of propagation patterns. This approach has the advantage of being completely noninvasive and readily deployable in any clinical setting without requiring specialized catheter placement.

### Imaging-Based Assessment Alternatives

The direction of electrical impulse propagation may be inferred by analyzing the motion of the lateral wall using various imaging techniques such as fluoroscopy, echocardiography, cardiac magnetic resonance imaging, direct observation during open procedures, or optical recording. As the electrical impulse propagates along the lateral wall, it causes mechanical contraction of that region. Comparing the contraction timing at various locations (such as at a location close to the base of the heart and a location close to the apex of the heart) provides information indicative of the direction of propagation of the electrical impulse along the lateral wall. Tissue Doppler imaging during echocardiography can assess regional contractility timing, while strain imaging can evaluate the sequence of myocardial deformation. Cardiac magnetic resonance tagging can provide a detailed assessment of regional wall motion. These imaging modalities complement electrical measurements and may be performed either during the procedure or subsequently during follow-up.

### Time Delay Measurement Methodologies

The time delay from septal activation to lateral wall activation provides a quantitative measure of conduction velocity and conduction system engagement. Multiple methods may be employed to measure this interval, offering different advantages and clinical settings of applicability:

### Method 1: Pacing Stimulus to Ecg R-wave Peak

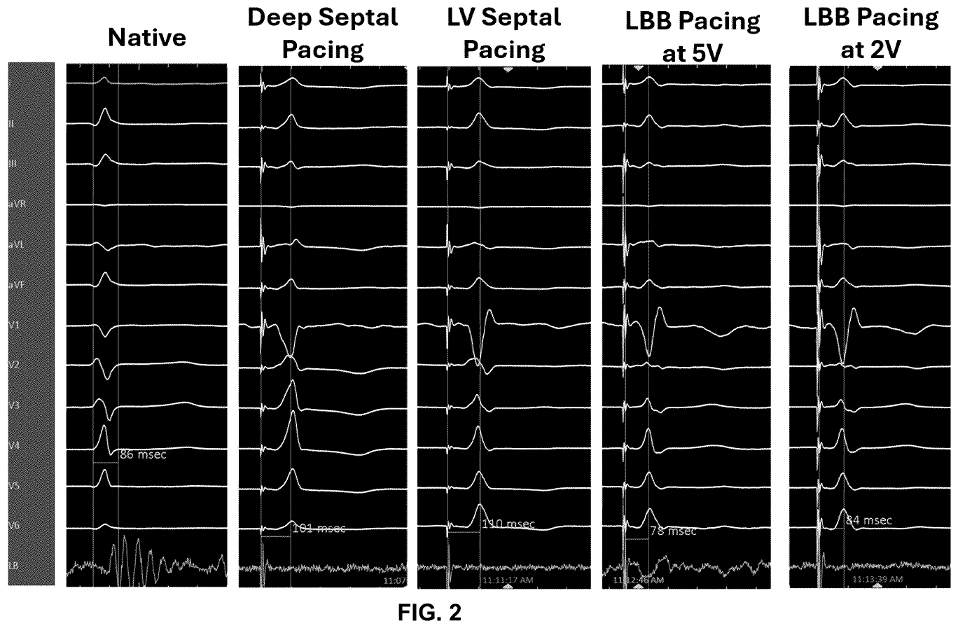

The time interval from the pacing stimulus delivered by the septal electrode to the peak of the R-wave in a selected electrocardiographic lead represents a macroscopic measurement of overall left ventricular activation time and is readily obtained from standard 12-lead electrocardiographic recordings. The R-wave peak represents maximal electrical activity and is easily identified on the electrocardiogram. This method provides low spatial resolution as it measures overall ventricular activation rather than lateral wall activation specifically, and it includes delay from epicardial-to-body surface propagation, not related to septal-to-lateral wall conduction. However, this method is simple, requires no specialized equipment, and is readily performed in any clinical setting. FIG. 2 shows appropriate R wave peak time measurements during septal electrode implantation at various depths. Measurements of 101-110-78-84 milliseconds reflect the present criteria for differentiation between different types of capture, as shown (note changes in QRS morphology that are also part of the acceptance criteria).

### Method 2: Pacing Stimulus to Earliest Lateral Wall Electrode Activation

The time interval from the pacing stimulus delivered by the septal electrode to the earliest detected electrical activation at the at least two lateral wall electrodes represents a direct measurement of conduction through either the specialized conduction system or myocardium, without delay from slower surface propagation. This measurement captures the actual conduction time from the septum to lateral wall tissue depolarization. This is the preferred method for precise assessment of conduction system engagement because it measures the specific anatomic region of interest and provides the most physiologically meaningful information.

### Method 3: Pacing Stimulus to Peak Lateral Wall Activation Signal

The time interval from the pacing stimulus delivered by the septal electrode to the peak electrical activation signal detected at the at least two lateral wall electrodes represents the time of maximal depolarization at each location. The peak of the activation signal may more accurately represent the time of maximal voltage change at each electrode than the initial deflection. This method offers intermediate precision between the earliest and only-surface measurements.

### Method 4: QRS Onset to Earliest Lateral Wall Activation

The time interval from the onset of the QRS complex on an electrocardiogram to the earliest detected electrical activation at the at least two lateral wall electrodes accounts for any baseline conduction delay before the septal pacing stimulus is delivered and is useful in assessing the impact of pacing on total left ventricular activation time. This measurement is particularly useful in patients with pre-existing conduction delay, allowing comparison of total activation time before and after pacing rather than just the interval from pacing stimulus.

Each method offers distinct advantages, and multiple methods may be employed in a single patient to provide complementary information and cross-validation of findings.

### Detection of Left Bundle Branch Capture

### Fundamental Method for Patients With Abnormal Baseline Activation

The present invention provides a method for detecting a capture of a left bundle branch of a heart for pacing purposes. The method comprises the following steps:

Step (a): Deploying a Septal Electrode. A septal electrode configured for left bundle branch pacing is deployed in an interventricular septum of a heart at a location intended for left bundle branch pacing. The electrode is typically positioned using fluoroscopic guidance, allowing visualization of the electrode position relative to the interventricular septum and surrounding cardiac structures. The septal electrode serves as the pacing cathode, with the pacemaker housing or an auxiliary return electrode serving as the anode. The electrode is advanced until it reaches a location where left bundle branch capture is thought possible based on anatomic landmarks, endocardial recordings, or mapping techniques.

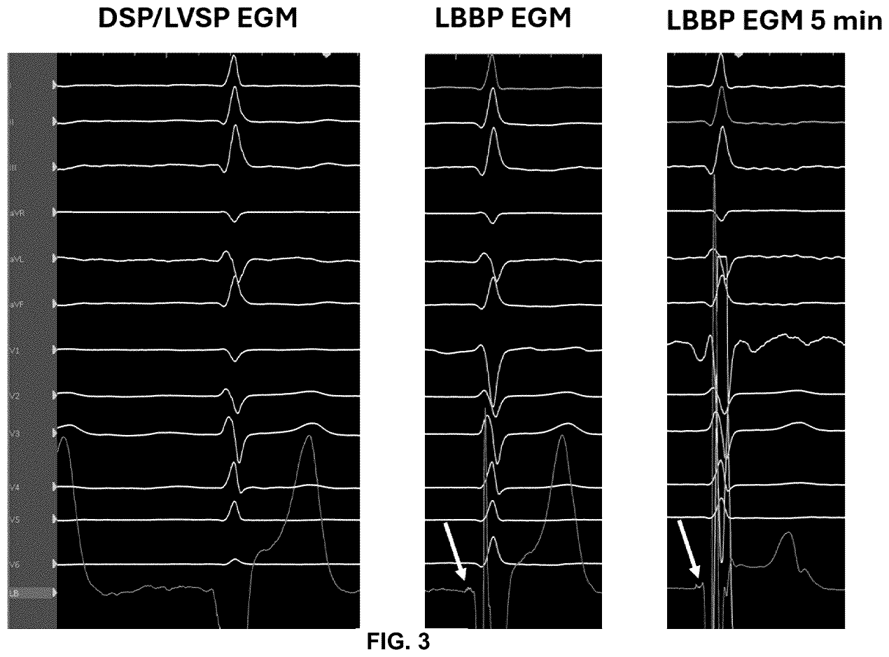

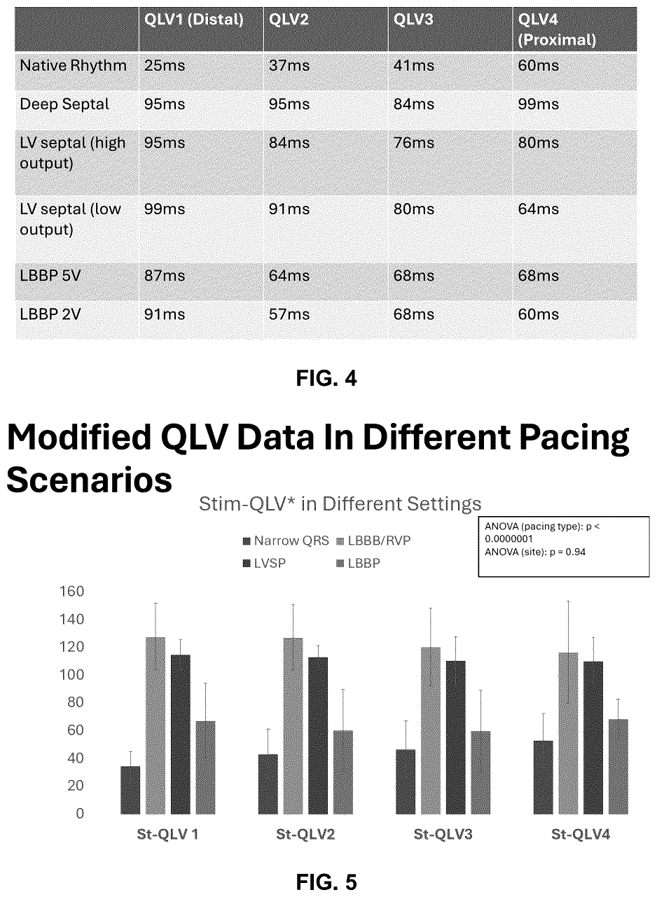

Step (b): Monitoring Activation Direction and Timing. The direction of electrical impulse propagation along the lateral wall during left ventricle activation is monitored, and the time delay from electrical activation of the interventricular septum to electrical activation of the lateral wall is measured. This baseline assessment occurs prior to initiation of pacing via the septal electrode and establishes the pre-pacing electrical characteristics. In patients with abnormal baseline conduction, such as those with left bundle branch block or right ventricular pacing-induced dyssynchrony, the baseline activation direction is typically from base to apex or in other non-physiological patterns such as side-to-side propagation, with prolonged activation delays typically in the range of 90-140 milliseconds. This baseline assessment establishes the reference point for comparison after pacing is initiated. FIG. 4 shows a table with the first line showing short local activation times measured at the quadripolar coronary sinus lead (QLV1-4) during native conduction with a narrow QRS, with the shortest time at the most distal apical pole—25 milliseconds, and the longest time at the most basal pole—60 milliseconds. This indicates a normal apex-to-base direction of activation. Subsequent rows show local activation times at the same poles during the progressive advancement of the septal electrode, indicating achievement of the Deep Septal, LV septal, and LBB capture positions (LBBP) at high and low pacing outputs (as labeled). Corresponding paced 12-lead ECG and intracardiac recordings are shown in FIGS. 2 and 3.

Step (c): Initiating Cardiac Pacing. Cardiac pacing is initiated using the septal electrode. The pacing may consist of a single pacing stimulus to assess acute response, a limited series of temporary pacing stimuli to observe the effect without committing to permanent pacing, or continuous release of pacing signals for sustained therapy. The pacing output, defined as both amplitude (voltage) and pulse width (duration), is typically set at or above the capture threshold for the septal location, ensuring reliable depolarization of the target tissue. Pacing is typically maintained long enough to observe steady-state activation patterns, allowing at least 2-5 cardiac cycles for the heart to reach a stable response.

Step (d): Identifying Left Bundle Branch Capture in Patients with Abnormal

Baseline. For patients with abnormal direction of electrical impulse propagation along the lateral wall of the left ventricle at baseline, left bundle branch capture is identified upon detecting:

First alternative criterion: A restoration or change toward a physiologically normal apex-to-base direction of electrical impulse propagation along the lateral wall after initiation of cardiac pacing. This indicates that the pacing stimulus has successfully engaged the left bundle branch and activated the lateral wall via rapid Purkinje conduction in the normal sequence from apical to basal regions. The reversal of propagation direction from abnormal baseline patterns to the physiologic apex-to-base pattern provides strong evidence of conduction system engagement.

Second alternative criterion: A shortening of the time delay from electrical activation of the interventricular septum to electrical activation of the lateral wall by at least 15 percent compared to baseline. This quantitative criterion ensures that modest, physiologically insignificant changes are not mistakenly interpreted as successful capture, while still detecting true restoration of conduction system engagement. For example, if the baseline delay was 100 milliseconds, a reduction to 85 milliseconds would satisfy this criterion. The 15 percent threshold has been selected based on clinical experience to provide appropriate specificity while maintaining sensitivity for true capture detection.

Either criterion may be satisfied individually, or both criteria may be present together, providing complementary confirmation of successful capture.

Step (e): Identifying Left Ventricular Septal Capture in Patients with Abnormal Baseline. For patients with abnormal direction of electrical impulse propagation along the lateral wall at baseline, left ventricular septal capture (defined as capture without left bundle branch engagement) is identified upon detecting:

First alternative criterion: Persistence or worsening of abnormal electrical impulse propagation direction after initiation of cardiac pacing. This indicates that the pacing stimulus has failed to engage the left bundle branch and instead depolarizes surrounding septal myocardium, which then conducts slowly at myocardial velocities of 0.3-0.4 meters per second to the lateral wall in the same abnormal pattern as occurred before pacing. Examples include continued base-to-apex propagation, side-to-side propagation, or other non-physiological patterns. Even if faster conduction velocities may be present in the left ventricular subendocardium, the normal impulse propagation will not be restored.

Second alternative criterion: Persistence of prolonged time delay from electrical activation of the interventricular septum to electrical activation of the lateral wall, wherein the time delay remains within 15 percent of the baseline delay or increases. For example, if baseline delay was 100 milliseconds, persistence of delays between 85-115 milliseconds or increases above 100 milliseconds indicates absence of rapid conduction via the specialized conduction system. This criterion indicates that the septal pacing has failed to restore rapid Purkinje conduction and instead continues myocardial propagation.

Persistence or worsening indicates that the septal pacing stimulus has depolarized myocardium without accessing the conduction system, allowing continued slow conduction similar to baseline. Again, even with faster conduction velocities of the left ventricular subendocardium, the longer time delays will persist.

### Enhanced Method for Patients With Normal Baseline Activation

For patients presenting with normal baseline direction of electrical impulse propagation along the lateral wall of the left ventricle from apex-to-base (such as patients with narrow baseline QRS duration less than 120 milliseconds or intact baseline conduction), the method further comprises:

Preservation Criterion: Detecting preservation of the direction of electrical impulse propagation, with continued apex-to-base direction after initiation of septal pacing. This indicates successful engagement of the left bundle branch, which maintains normal physiology.

Absence of Delay Increase Criterion: Detecting an absence of an increase of a delay between activation of the interventricular septum and the lateral wall by more than 10 percent of a baseline delay. This criterion is more stringent than the 15 percent threshold used for abnormal baseline patients because patients with normal baseline conduction may not tolerate even modest increases in activation delays.

Alternatively, it is not uncommon, particularly in patients with advanced conduction system disease, to have a significant latency from the pacing stimulus prior to capture of the conduction system. In this situation, the timing between the pacing spike and lateral wall activation may be long, but it is still possible for the conduction system capture to have occurred. To overcome this pattern, a delay between the local activation-measured from the electrogram on the lead rather than from the pacing stimulus to lateral wall activation-may be a better way of assessing bidirectional activation time.

This embodiment recognizes that in patients with normal baseline conduction, successful left bundle branch capture should maintain the normal activation sequence without introducing a significant additional delay.

### Enhanced Quantitative Criteria for Specific Diagnosis

For Identifying Left Bundle Branch Capture: The method may further comprise identifying left bundle branch capture by detecting at least one of the following quantitative criteria:

First quantitative criterion: Shortening of the time delay from baseline measurement by at least 25 percent. This represents a substantial and robust restoration of rapid conduction via the specialized conduction system. For example, a reduction from 120 milliseconds to 90 milliseconds represents 25 percent shortening and meets this criterion. This more stringent 25 percent criterion provides additional diagnostic confidence compared to the 15 percent criterion.

Second quantitative criterion: A time delay from the pacing stimulus to left ventricular lateral wall activation of less than 80 milliseconds. This absolute value corresponds to rapid conduction through Purkinje fibers at normal velocities of 2-3 meters per second across typical ventricular distances of 160-240 millimeters. More preferably, the time delay should be less than 75 milliseconds, indicating particularly robust Purkinje engagement. Most preferably, the time delay should be less than 70 milliseconds, indicating optimal rapid conduction.

Either criterion may be satisfied individually, or both may be present, providing increasing diagnostic confidence.

For Identifying Left Ventricular Septal Capture: The method may further comprise identifying left ventricular septal capture by detecting at least one of the following quantitative criteria:

First quantitative criterion: The direction of electrical impulse propagation along the lateral wall continues to be abnormal after pacing. This includes but is not limited to base-to-apex propagation, side-to-side propagation, or other non-physiological patterns. This directly indicates failure of septal pacing to engage the conduction system.

Second quantitative criterion: A time delay from the pacing stimulus to left ventricular lateral wall activation exceeding 85 milliseconds. This indicates slow myocardial conduction without engagement of the specialized conduction system. More preferably, the time delay should exceed 90 milliseconds, providing additional diagnostic confidence. Most preferably, the time delay should exceed 95 milliseconds, indicating definitively slow conduction. This measurement can be confounded by latency; wherein a method to overcome this is described above (Absence of delay increase criterion).

These quantitative thresholds provide clear diagnostic boundaries with minimal overlap between LBB capture and septal capture populations, enabling standardized application across different patient populations and operators.

### Alternative Measurement Approaches

The method of the present invention may employ measurements using at least two lateral wall electrodes configured to detect activation of a lateral wall of a left ventricle of the heart. In one embodiment, step (d) is performed by comparing electrical impulse arrival times and respective locations of the at least two lateral wall electrodes spaced along the lateral wall of the left ventricle. The sequence of arrival times indicates the direction of propagation. In another embodiment, step (b) is performed using a quadripolar lateral wall electrode placed in a coronary sinus along the lateral wall of the left ventricle of the heart. The quadripolar electrode (four electrodes) provides enhanced spatial resolution and allows more precise determination of propagation patterns.

In an alternative embodiment, step (b) may be performed using simultaneous electrocardiogram recordings obtained from a plurality of skin electrodes placed on a chest. Multi-lead electrocardiography provides a completely noninvasive assessment without requiring intracardiac electrode placement. The recording should include at least 12 standard leads, preferably 18-64 leads, to provide adequate spatial resolution for detecting propagation direction changes and timing variations.

### Clinical Context: Single Stimulus Versus Repeated Pacing Assessment

The method may employ a single pacing stimulus to assess the acute response, allowing observation of how the heart responds to a single septal stimulus. This approach minimizes procedural time but provides only a single data point. Alternatively, the method may employ a limited series of temporary pacing stimuli (typically 5-10 stimuli at a given electrode location) to assess the response pattern and confirm that observed changes represent true capture rather than spontaneous variation. This approach provides more robust data at the cost of slightly increased procedure duration. Continuous pacing at a fixed rate (such as 60-80 beats per minute) provides steady-state activation patterns and is appropriate for final confirmation and permanent device programming. Both approaches can be performed at different pacing outputs to minimize the influence of the size of the virtual electrode on the conduction velocity.

### Bidirectional Pacing Protocol for Differentiating Capture

### Physiological Foundation for Bidirectional Measurement

A novel and powerful method for differentiating left bundle branch capture from septal myocardial capture employs bidirectional pacing protocols that exploit the preferential anterograde conduction properties of the specialized His-Purkinje system. The His-Purkinje system exhibits high-velocity anterograde (physiological direction) conduction at 2 to 3 meters per second when impulses propagate from the His bundle through the bundle branches and Purkinje fibers to the ventricular myocardium. However, retrograde conduction from ventricular myocardium back into the proximal specialized conduction system faces significant impedance and functional barriers.

This asymmetry arises from several mechanisms. First, the Purkinje-myocardial junction exhibits directional differences in conduction delay due to the geometry of the junction and the differences in cellular properties between Purkinje fibers and myocardium. Retrograde transmission across this junction is impeded by refractory properties and decremental conduction, where impulses propagating backward encounter progressively increasing refractoriness and conduction delay. Second, the specialized conduction tissue typically exhibits unidirectional block or preferential conduction in the physiological direction due to the directional properties of gap junctions and the cellular arrangement within the conduction tissue. The arrangement of intercalated discs and gap junctions creates preferential conduction from proximal to distal tissues. Third, the large electrical load presented by ventricular myocardium when conducting retrogradely into the narrow specialized fibers creates source-sink mismatch. The large mass of ventricular myocardium attempting to depolarize the small volume of specialized tissue encounters electrical impedance that prevents effective retrograde propagation into the conduction system.

This asymmetry creates a distinctive conduction pattern: when a septal electrode has successfully captured the left bundle branch, electrical impulses conduct rapidly in the anterograde direction through the specialized conduction system to the lateral wall, resulting in short conduction times of 40-60 milliseconds. In contrast, when stimulating from the lateral wall electrodes, impulses must propagate slowly through ventricular myocardium at velocities of 0.3-0.4 meters per second and cannot effectively enter the specialized conduction system retrogradely, resulting in much longer conduction times of 80-100 milliseconds or greater to reach the septal electrode. The difference between these conduction times, termed the bidirectional conduction time differential or “delta,” provides a powerful functional assessment of conduction system engagement.

In contrast, when septal pacing engages only ventricular myocardium without conduction system engagement, conduction is slow in both directions through myocardial tissue at 0.3-0.4 meters per second, resulting in long conduction times bidirectionally with minimal difference between forward and reverse directions. Both anterograde and retrograde conduction times fall in the 80-100 millisecond range, resulting in a delta of only 5-15 milliseconds.

### Complete Bidirectional Pacing Method

The method for differentiating between left ventricular septal capture and left bundle branch capture during heart pacing comprises the following steps:

Step (a): Deploying Septal and Lateral Wall Electrodes. A septal electrode configured for left bundle branch pacing is deployed in an interventricular septum of a heart at a location intended for left bundle branch pacing. Additionally, at least two lateral wall electrodes are deployed along a lateral wall of a left ventricle, wherein the at least two lateral wall electrodes are spaced apart along a direction between an apex and a base of the heart. The lateral wall electrodes serve as bidirectional sensing and pacing sites. The spaced positioning ensures that electrodes span the apex-to-base dimension, providing adequate spatial separation to assess conduction properties.

Step (b): Establishing Baseline Measurements. Prior to the bidirectional pacing protocol, baseline measurements may be obtained with the septal electrode at rest or during intrinsic rhythm, establishing reference values for subsequent comparison.

Step (c): First Pacing Protocol in Anterograde Direction. A first pacing protocol is initiated by pacing from the septal electrode and measuring a first conduction time from a pacing stimulus delivered by the septal electrode to electrical activation detected at the at least two lateral wall electrodes. This represents “anterograde” direction pacing, proceeding from septal toward lateral wall tissue. The first conduction time typically reflects conduction through rapid Purkinje fibers if the left bundle branch is captured, typically resulting in times of 40-60 milliseconds. If only the septal myocardium is captured, the first conduction time reflects slow myocardial conduction and typically ranges from 80-100 milliseconds or longer.

Step (d): Second Pacing Protocol in Retrograde Direction. A second pacing protocol is initiated by pacing from at least one of the at least two lateral wall electrodes and measuring a second conduction time from a pacing stimulus delivered by said at least one lateral wall electrode to electrical activation detected at the septal electrode. This represents “retrograde” direction pacing, proceeding from the lateral wall toward the septum. This is the opposite direction from physiologic conduction. The second conduction time typically reflects myocardial conduction in both capture scenarios because retrograde conduction through the specialized system is blocked or severely impeded. The second conduction time typically ranges from 80-100 milliseconds or longer in both LBB and septal capture scenarios.

Step (e): Calculating Bidirectional Conduction Time Differential. A bidirectional conduction time differential (delta) is calculated as an absolute difference between the first conduction time (anterograde) and the second conduction time (retrograde). This delta measurement quantifies the directional asymmetry in conduction times and provides the diagnostic criterion. The delta is calculated as follows: Delta=absolute value of (First Conduction Time minus Second Conduction Time). For example, if the first conduction time is 50 milliseconds and the second conduction time is 95 milliseconds, then Delta equals 45 milliseconds.

Step (f): Identifying Left Bundle Branch Capture. Left bundle branch capture is identified when the bidirectional conduction time differential exceeds a threshold value. Specifically, the first conduction time is shorter than the second conduction time (for example, 45 milliseconds versus 95 milliseconds, delta equals 50 milliseconds). This pattern indicates rapid anterograde conduction through the specialized left bundle branch conduction system when pacing from the septal electrode, and slower retrograde myocardial conduction when pacing from the lateral wall electrodes. This asymmetry in conduction times is the hallmark of conduction system capture.

The threshold value is selected from the range of 11 milliseconds to 50 milliseconds to account for individual variation in conduction properties. The preferred range is 15 milliseconds to 35 milliseconds, accounting for most clinical scenarios while maintaining appropriate specificity. The most preferred range is 20 milliseconds to 30 milliseconds, providing an optimal balance between sensitivity and specificity. These thresholds are calibrated to distinguish the directional asymmetry of specialized tissue, which creates large deltas, from the symmetric slow conduction of myocardium, which creates small deltas.

Step (g): Identifying Left Ventricular Septal Capture. Left ventricular septal capture is identified when the bidirectional conduction time differential is less than the threshold value. In this scenario, the first and second conduction times are similar (for example, first conduction time 85 milliseconds, second conduction time 95 milliseconds, delta equals 10 milliseconds). The delta may be expressed in absolute terms (milliseconds) or in relative terms, for example, as a percentage of the first or second conduction time, as the invention is not limited in this regard.

This pattern indicates comparable slow myocardial conduction in both pacing directions and the absence of specialized conduction system engagement. The symmetric timing in both directions is characteristic of purely myocardial conduction.

### Quadripolar Electrode Lead Implementation

In a preferred embodiment, the bidirectional pacing method is implemented using a quadripolar electrode lead. This standardized approach improves reproducibility and provides enhanced diagnostic information compared to simpler two-electrode systems.

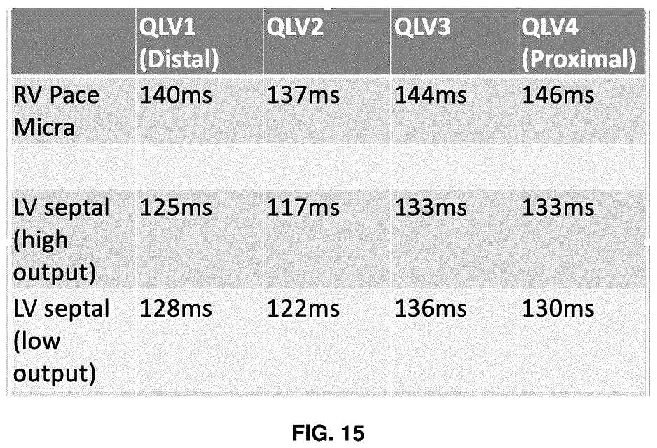

Electrode Configuration. The at least two lateral wall electrodes comprise a quadripolar electrode lead with four electrodes numbered 1 through 4, positioned with electrode 1 closest to the apex and electrode 4 closest to the base, as seen in FIGS. 2 and 15. This standardized configuration ensures reproducible electrode positioning and simplifies interpretation of results. The electrodes are typically spaced 10-15 millimeters apart along the long axis of the ventricle.

Anterograde Pacing and Measurement. Step (c) further comprises measuring the first conduction time from the septal electrode to each of electrodes 1, 2, 3, and 4 individually. This provides four separate conduction time measurements and allows identification of the pattern of lateral wall activation. In LBB capture, the first conduction times should be relatively short and similar across all four electrodes, reflecting rapid Purkinje conduction reaching the entire lateral wall with minimal delay variation. In septal capture, the first conduction times should be relatively long and show progressive delay from proximal to distal electrodes, reflecting progressive myocardial conduction.

FIG. 5 shows a summary of measurements in 17 patients with different baseline conduction disturbances and different types of capture achieved. Abnormally late activation of the lateral LV wall is present at baseline in patients with Left Bundle Branch block (LBBB) and RV pacing (RVP) compared to narrow QRS (normal conduction). Highly statistically significant differences are present between the subgroups of LBB capture (LBBP) and left septal capture (LVSP), reflecting engagement of the conduction system with LBBP and abnormal slow conduction with LVSP. Differentiation between LBBP and LVSP based on standard criteria is frequently ambiguous. Lateral wall activation measurements allow solving this diagnostic dilemma.

Retrograde Pacing and Measurement. Step (d) further comprises pacing sequentially from each of electrodes 1, 2, 3, and 4 individually and measuring the second conduction time from each respective electrode to the septal electrode. This systematic approach captures retrograde conduction time for each combination of lateral wall electrode and septal electrode. Typically, the second conduction times are relatively uniform across all four electrodes, reflecting slow retrograde myocardial propagation that is relatively unaffected by electrode location.

Enhanced Diagnostic Criteria for LBB Capture. Step (f) further comprises identifying left bundle branch capture when all of the following criteria are simultaneously present:

Criterion 1: The bidirectional conduction time differential exceeds the threshold value for at least two of the four electrodes. This requirement ensures that the observed asymmetry is consistent across multiple lateral wall sites and is not a single anomalous electrode reading or measurement artifact. The “at least two of four” criterion provides robustness against isolated electrode malfunction.

Criterion 2: The first conduction time measured from the septal electrode to electrodes 1, 2, 3, and 4 shows minimal variation, specifically less than 15 milliseconds difference between the earliest and latest activation among the four electrodes. This low variation indicates rapid and synchronous activation of the entire lateral wall via the specialized conduction system. This synchronous pattern is characteristic of Purkinje fiber activation, where the entire lateral wall is reached almost simultaneously through the network of rapidly conducting fibers. In contrast, slow myocardial conduction would produce progressive delay from proximal to distal electrodes with much greater variation. As can be appreciated by a person skilled in the art, the exact measurement and difference will depend on the actual distance between the electrodes, as larger distances may result in a more pronounced time delay.

Criterion 3: The second conduction time measured when pacing from any of electrodes 1, 2, 3, or 4 to the septal electrode shows longer delays exceeding the first conduction time by at least the threshold value. This indicates slow myocardial conduction, which is unable to effectively engage the specialized conduction system in the retrograde direction. This occurs due to the unidirectional conduction properties of Purkinje fibers, which preferentially conduct in the physiologic anterograde direction.

The requirement for all three criteria to be simultaneously present ensures that the diagnosis of LBB capture is robust and not based on a single parameter that might be affected by measurement error or anatomic variation.

### Patient-Specific Threshold Adjustment

The threshold value for the bidirectional conduction time differential may be dynamically adjusted based on individual patient factors, recognizing that baseline conduction properties vary among patients based on underlying electrophysiology and structural disease.

Baseline QRS Duration. The intrinsic QRS duration in the patient's baseline intrinsic rhythm reflects the overall conduction system properties and myocardial conduction times. Patients with prolonged baseline QRS have established conduction abnormalities and slower overall conduction and may require adjustment of thresholds upward to account for inherently slower conduction velocities. Conversely, patients with narrow QRS have normal baseline conduction and may benefit from lower thresholds to detect even subtle failure of conduction system engagement.

Presence or Absence of Left Bundle Branch Block. Patients with baseline left bundle branch block have established conduction system pathology with documented failure of one bundle branch. These patients have proven conduction system disease and may have altered properties affecting bidirectional conduction times. The expected delta in a patient with baseline LBBB undergoing LBBAP may differ from that of a patient with normal baseline conduction.

Presence or Absence of Ischemic Heart Disease or Myocardial Scarring. Myocardial infarction with scarring or ischemia alters myocardial conduction velocities and pathways by slowing propagation through diseased tissue. Scarred tissue conducts more slowly than normal myocardium, potentially slowing all conduction times and affecting the magnitude of the delta. Patients with extensive scarring might show smaller deltas than would be expected in patients with normal myocardium, even with true LBB capture.

Left Ventricular Ejection Fraction. The degree of ventricular dysfunction may correlate with altered conduction properties due to chamber dilation, wall motion abnormality, or underlying fibrosis. Though this is an indirect marker of conduction properties, patients with very low ejection fraction may have different conduction characteristics than those with preserved ejection fraction.

### Specific Threshold Recommendations Based on Patient Factors:

For patients with left bundle branch block or wider baseline QRS duration greater than 140 milliseconds, utilize a higher threshold value of 25 to 40 milliseconds. These patients have proven conduction system disease and slower overall conduction. A higher threshold accounts for correction of baseline conduction block/asymmetry characteristic of overcoming the conduction block via conduction system engagement.

These patient-specific adjustments recognize the heterogeneity of patient populations while maintaining diagnostic accuracy.

### Validation Across Multiple Pacing Output Voltages

The bidirectional pacing method may further comprise validation of left bundle branch capture by:

Multiple Voltage Measurements. Performing the bidirectional pacing measurements at multiple pacing output voltages ranging from threshold capture voltage to a voltage at least 2-fold higher than the threshold capture voltage. The capture threshold is defined as the minimum voltage (with fixed pulse width, typically 0.4 milliseconds) necessary to reliably capture the target tissue. Higher voltages may recruit a larger volume of tissue or engage different tissue structures as stimulation intensity increases. Testing at multiple voltages determines whether capture is stable and does not depend critically on voltage selection/virtual electrode size (FIG. 4 example illustrates stable LBB capture, independent of output, rows 5-6, LBBP at 5 and 2 Volts).

Confirming Stable Capture. Confirming left bundle branch capture when the bidirectional conduction time differential remains consistently above the threshold value across all tested pacing output voltages. This consistency indicates stable capture of the specialized conduction system that does not depend on specific pacing parameters. If the delta remains suprathreshold at threshold voltage and also at 2-fold threshold voltage and at intermediate voltages, this provides strong evidence of true conduction system engagement that is robust and will not be lost due to minor changes in device output or changes in pacing threshold over time.

Detecting Loss of Capture or Unstable Capture. Identifying loss of left bundle branch capture or unstable capture when the bidirectional conduction time differential decreases below the threshold value at lower pacing outputs. This observation suggests that at lower voltages, capture is limited to the myocardium, while at higher voltages, some conduction system engagement occurs. This is an unstable and undesirable situation, indicating that the electrode is positioned at the margin between myocardial and conduction system tissue, and capture may be lost with slight increases in threshold. In this case, electrode repositioning should be considered.

### Clinical Application: Therapy Delivery Decision Algorithm

### Decision-Making Process Using Bidirectional Pacing

The bidirectional pacing method may be applied to guide acute therapy delivery decisions during the implantation procedure, determining whether to proceed with septal pacing only or to utilize biventricular pacing. A method of delivering a pacing therapy to a heart comprises:

Step (a): Septal Electrode Deployment. a Septal Electrode Configured for Left bundle branch pacing is deployed in an interventricular septum of a heart at a location intended for left bundle branch pacing. The electrode is advanced to a position where left bundle branch capture is anticipated based on anatomic considerations.

Step (b): Lateral Wall Electrode Deployment. At least two lateral wall electrodes are deployed along a lateral wall of a left ventricle, providing the capability for bidirectional pacing and sensing. These electrodes ideally should span the apex-to-base dimension to allow assessment of propagation patterns.

Step (c): Left Bundle Branch Capture Detection. Left bundle branch capture is detected by performing: (i) initiation of heart pacing using the septal electrode, and (ii) application of the bidirectional pacing method to confirm left bundle branch capture. The bidirectional conduction time differential is calculated and compared to the patient-specific threshold that has been established based on the patient's baseline QRS duration, presence of LBBB, myocardial scarring, and left ventricular ejection fraction.

Step (d): Continue Septal Pacing if LBB Captured. If the left bundle branch is captured, as confirmed in step (c), meaning the bidirectional conduction time differential exceeds the patient-specific threshold, and all other confirmatory criteria are met, the pacing therapy continues to be delivered to the heart using the septal electrode exclusively. The septal electrode is secured in place. The patient receives the benefits of physiologic conduction system pacing with minimal lead complexity, single-site pacing, and the most favorable long-term outcomes.

Step (e): Switch to Biventricular Pacing if LBB Not Captured. If the left bundle branch cannot be captured as confirmed in step (c), meaning the bidirectional conduction time differential remains below the patient-specific threshold or other diagnostic criteria are not satisfied, biventricular heart pacing is initiated using at least the septal electrode and at least one lateral wall electrode. Biventricular pacing provides resynchronization therapy as an alternative when conduction system capture cannot be achieved. The biventricular pacing strategy allows optimization of ventricular synchrony through coordinated pacing of two separate sites, septal and lateral wall, rather than relying on a single pacing site. Optimal atrioventricular and interventricular delays are programmed.

This decision tree ensures that patients receive the most appropriate pacing strategy based on the actual electrophysiologic findings determined by objective measurement, avoiding the clinical failure of accepting inadequate septal myocardial capture when better alternatives are available.

### Chronic Monitoring for Loss of Left Bundle Branch Capture

### Long-term Capture Verification and Loss Detection

A method of monitoring left bundle branch capture during chronic heart pacing using a septal electrode positioned in an interventricular septum comprises:

Step (a): Periodic or Continuous Monitoring. Performing periodic or continuous monitoring of at least one of the following parameters:

First monitored parameter: A direction of electrical impulse propagation along a lateral wall of a left ventricle using implanted lateral wall electrodes or noninvasive electrocardiographic mapping. The baseline apex-to-base direction established at implantation should be maintained if capture is preserved. Any reversion to base-to-apex or other abnormal patterns indicates loss of capture. Monitoring may occur automatically during device interrogations, which typically occur every 3-12 months, or more frequently if indicated by remote monitoring capabilities.

Second monitored parameter: A time delay from electrical activation of the interventricular septum to electrical activation of the lateral wall. The baseline timing established at implantation (typically 40-80 milliseconds during successful LBB capture) should remain stable. Significant prolongation by more than 20 percent compared to baseline indicates loss of capture. For example, if the implant-time delay was 60 milliseconds, an increase to greater than 72 milliseconds would indicate deterioration.

Third monitored parameter: A bidirectional conduction time differential measured by alternating pacing from the septal electrode and from lateral wall electrodes using the bidirectional pacing method described above. Maintenance of suprathereshold delta (above the patient-specific threshold) indicates continued conduction system engagement. A decrease below threshold indicates loss of capture.

The monitoring may be automated, performed at scheduled device check-ins, or triggered by patient symptoms or device diagnostics indicating possible pacing dysfunction. Modern implanted devices can perform automated measurements and alert clinicians to abnormalities.

Step (b): Detection of Loss of Left Bundle Branch Capture. Loss of left bundle branch capture is detected upon identifying at least one of the following:

First detection criterion: A change in the direction of electrical impulse propagation from apex-to-base (normal) to a non-apex-to-base pattern, such as reverting to base-to-apex or developing side-to-side propagation. This change indicates loss of physiological activation sequence and is consistent with loss of conduction system engagement and return to myocardial pacing.

Second detection criterion: An increase in the time delay by more than 20 percent compared to a baseline measurement obtained when left bundle branch capture was confirmed at implantation. At least a 20 percent or higher threshold prevents false positives from normal fluctuations while detecting clinically significant deterioration. For example, if baseline delay at implantation was 50 milliseconds, an increase to greater than 60 milliseconds would trigger detection of loss. A 20 percent threshold or higher accommodates normal beat-to-beat variation while still detecting clinically important changes.

Third detection criterion: A decrease in the bidirectional conduction time differential to below the patient-specific threshold value. This indicates loss of specialized conduction system engagement and transition to myocardial capture. If the delta decreases from 35 milliseconds at implantation to 15 milliseconds during follow-up, and the patient-specific threshold is 25 milliseconds, this would trigger loss detection.

Step (c): Corrective Actions Upon Loss Detection. Upon detecting loss of left bundle branch capture, at least one corrective action is initiated, selected from a menu of options:

First corrective action: Alerting a patient or healthcare practitioner through implanted device notifications or patient-directed alerts in a remote monitoring system. Immediate notification allows clinical evaluation and intervention planning. The patient may be instructed to seek urgent evaluation or the healthcare practitioner notified to schedule a clinic visit.

Second corrective action: Adjusting pacing parameters, including increasing output voltage to restore capture if loss is due to a rise in pacing threshold. The capture threshold may increase over time due to fibrosis around the electrode. Increasing voltage above the new threshold or changing to unipolar from bipolar pacing (if originally programmed bipolar) may restore capture. Alternatively, pulse width may be increased, or the pacing site may be changed to an alternative electrode on the lead if available.

Third corrective action: Automatically switching to biventricular pacing by the implanted device. The device activates pacing from lateral wall electrodes in addition to septal pacing to maintain adequate resynchronization. This ensures continued resynchronization therapy even if LBB capture cannot be maintained.

Fourth corrective action: Scheduling a follow-up evaluation or device interrogation. Comprehensive assessment including electrocardiography, device interrogation to measure capture threshold and impedance, and possibly imaging to determine if lead migration or fracture has occurred. This evaluation determines the cause of capture loss and identifies the optimal remedial strategy.

### System Architecture for Chronic Monitoring

A comprehensive system for detecting left bundle branch capture acutely and monitoring its persistence chronically comprises:

Component (a): Septal Electrode. A septal electrode configured for deployment in an interventricular septum and delivery of pacing stimuli. The electrode typically consists of a cathode (stimulating tip) and may have integrated or separate electrodes for sensing. The electrode may be a fixed-curve electrode designed to position within the interventricular septum, or a more flexible lead that adapts to septal anatomy.

Component (b): Lateral Wall Electrodes. At least two lateral wall electrodes configured for deployment along a lateral wall of a left ventricle and spaced apart along a direction between an apex and a base of the heart. These electrodes are configured to sense electrical activation of the lateral wall and to deliver pacing stimuli for retrograde conduction measurement during bidirectional pacing assessment (or for biventricular pacing, if needed). The lateral wall electrodes provide sensing capability for monitoring activation patterns and pacing capability for the bidirectional protocol.

Component (c): Pacing Module. A pacing module configured to deliver pacing stimuli from the septal electrode at adjustable pacing outputs, including adjustable amplitude (voltage) and pulse width (duration). The pacing module also delivers pacing stimuli from the at least one lateral wall electrode for retrograde conduction measurement. The pacing module manages the sequencing and timing of pacing stimuli for both anterograde (septal to lateral) and retrograde (lateral to septal) measurements. It can be programmed to deliver different pacing protocols as needed.

Component (d): Sensing Module. A sensing module configured to receive electrical activation signals from the at least two lateral wall electrodes when pacing from the septal electrode. The sensing module also receives electrical activation signals from the septal electrode when pacing from the at least two lateral wall electrodes. The sensing module detects the precise timing of electrical activation at each electrode and transmits these measurements to the processing module. The sensing module includes filtering, amplification, and analog-to-digital conversion to extract timing information from raw electrograms.

Component (e): Processing Module. A processing module configured to perform multiple processing tasks:

First function: Determine a sequence of electrical activation among the at least two lateral wall electrodes to identify a direction of electrical impulse propagation (apex-to-base versus base-to-apex or other patterns). The processing module compares activation times at each electrode to determine which electrode is activated first, second, etc.

Second function: Measure a first conduction time from pacing stimuli delivered by the septal electrode to electrical activation detected at the at least two lateral wall electrodes. The processor identifies the timing of the pacing stimulus and the timing of electrical activation at each lateral wall electrode and calculates the interval.

Third function: Measure a second conduction time from pacing stimuli delivered by the at least two lateral wall electrodes to electrical activation detected at the septal electrode. The processor performs similar timing measurements in the retrograde direction.

Fourth function: Calculate a bidirectional conduction time differential (delta) as an absolute difference between the first conduction time and the second conduction time.

Fifth function: Compare the bidirectional conduction time differential to a patient-specific threshold value that has been established at implantation or can be dynamically adjusted based on patient factors. The processor compares the calculated delta to the threshold.

Component (f): Output Module. An output module configured to indicate capture status:

First indication: Indicate left bundle branch capture when the bidirectional conduction time differential exceeds the threshold value AND the direction of electrical impulse propagation is apex-to-base. This represents the desired outcome.

Second indication: Indicate left ventricular septal capture when the bidirectional conduction time differential is less than the threshold value OR the direction of electrical impulse propagation is non-apex-to-base. This indicates suboptimal capture requiring intervention.

The output module may generate alerts to clinicians and patients, generate reports documenting capture status, trigger corrective actions within the device, or notify healthcare practitioners as appropriate.

### Real-time Verification Using Electrocardiographic Imaging

### ECGi Fundamentals and Technological Capabilities

Electrocardiographic imaging (ECGi) represents a significant advancement in noninvasive cardiac electrophysiology that may be deployed for real-time verification of left bundle branch capture during septal electrode implantation. ECGi uses a large number of body surface electrodes, numbering at least 64 but frequently 120 to 256 electrodes, combined with three-dimensional cardiac geometry obtained from computed tomography or magnetic resonance imaging. Through these components, ECGi reconstructs epicardial and endocardial electrical activation patterns using computational inverse solution of the electrocardiographic problem. This sophisticated computational approach provides far greater spatial resolution than conventional 12-lead electrocardiography and can localize arrhythmic foci or activation sequences with accuracy better than 10 millimeters when compared to invasive mapping techniques.

ECGi systems provide isochrone maps displaying the temporal sequence of ventricular activation. These maps allow visualization of conduction pathways and identification of sites of earliest and latest activation. The color-coded maps show successive time points during ventricular depolarization, with clear visualization of the propagation direction and velocity of electrical activation throughout the ventricle. Multiple isochrone maps at 5-10 millisecond time intervals create an animation showing electrical propagation sweeping across the ventricular surface.

### Real-Time ECGi-Guided Implantation Procedure

Recent technological developments have enabled real-time or near-real-time ECGi deployment during cardiac procedures, including septal electrode implantation. This real-time capability provides immediate visual feedback to the implanting physician, allowing objective assessment of electrical synchronization as the septal electrode is advanced to different depths within the interventricular septum. The operator can observe the change in activation patterns as the electrode moves deeper into the septum, identifying the depth at which the pattern transitions from septal myocardial to left bundle branch pacing.

A method of real-time verification of left bundle branch capture during septal electrode implantation comprises:

Step (a): ECGi System Deployment. An electrocardiographic imaging (ECGi) system comprising a plurality of body surface electrodes is deployed prior to or during the implantation procedure. Typically, 64 to 256 electrodes are distributed around the patient's torso in a standardized array, providing comprehensive coverage of the surface from which electrical signals propagate. The electrodes may be arranged in a vest-like configuration or as individual electrodes placed on the torso. The electrode positions are recorded using three-dimensional position tracking.

Step (b): Acquiring Three-Dimensional Cardiac Geometry. A three-dimensional cardiac geometry is acquired from computed tomography or magnetic resonance imaging performed pre-procedurally, or alternatively, using an artificial intelligence-derived geometric model. The cardiac geometry establishes the anatomic framework upon which electrical activation patterns are projected. Pre-procedural computed tomography or magnetic resonance imaging provides patient-specific anatomic detail and high spatial resolution. Alternatively, artificial intelligence-derived models create a patient-specific cardiac geometry estimate based on body surface electrode positions, simple anthropometric measurements, and computational algorithms trained on large imaging databases. These imageless approaches offer the advantage of not requiring pre-procedural imaging studies and avoiding procedural delay.

Step (c): Recording Electrical Signals Continuously. Electrical signals are continuously recorded from a plurality of body surface electrodes during the implantation procedure. These surface potentials contain all the information necessary to reconstruct cardiac electrical activation when combined with the cardiac geometry through inverse computational methods. The continuous recording captures electrical activity throughout the implantation procedure as the electrode is advanced to different septal depths.

Step (d): Generating Real-Time Activation Maps. Real-time or near-real-time activation maps are generated showing ventricular electrical activation patterns as the septal electrode is advanced into the interventricular septum. Advanced computation allows these maps to be generated and displayed within 2-5 seconds of each test pacing maneuver, providing meaningful real-time feedback to the implanting operator. The operator views the activation maps on a display monitor adjacent to the fluoroscopy monitor, allowing simultaneous visualization of anatomic position (on fluoroscopy) and electrical activation (on ECGi maps).

Step (e): Performing Test Pacing at Multiple Depths. Test pacing from the septal electrode is initiated at multiple depths of septal penetration, and corresponding activation maps are generated for each pacing maneuver. This systematic approach allows the operator to observe how the pattern of electrical activation changes as the electrode moves deeper into the septum. At each depth, 5-10 pacing stimuli are delivered, and the corresponding ECGi-derived activation maps are computed and displayed.

Step (f): Comparing Activation Maps to Identify Transition. The activation maps obtained at different septal depths are compared to identify a transition from one pattern to another:

First pattern, left ventricular septal pacing: Characterized by lateral left ventricular activation occurring with delayed timing of 90-120 milliseconds from the pacing stimulus. The activation sequence is abnormal, such as base-to-apex direction or non-physiologic patterns. Activation propagates progressively from the septum outward through the myocardium at slow conduction velocities of 0.3-0.4 meters per second. On the activation map, the color codes would show a progressive “wave” of activation sweeping from the septal region outward.

Second pattern, left bundle branch pacing: Characterized by early septal activation followed by rapid and synchronous left ventricular lateral wall activation with restoration of physiological apex-to-base activation and shortened left ventricular activation time of 60-80 milliseconds from pacing stimulus. On the activation map, the color codes would show nearly simultaneous activation of the entire lateral wall with a characteristic pattern consistent with Purkinje fiber activation.

The identification of this transition point provides clear visual evidence of the depth at which conduction system engagement begins.

Step (g): Confirming Successful Left Bundle Branch Capture. Successful left bundle branch capture is confirmed when the activation map demonstrates the left bundle branch pacing pattern (apex-to-base direction, rapid synchronous lateral wall activation) AND left ventricular activation time is shortened by at least 15 percent compared to baseline measurements (pre-pacing) obtained at the beginning of the procedure. Alternatively, left ventricular activation time should be shortened by at least 15 percent compared to more superficial septal pacing sites tested earlier. This 15 percent threshold ensures clinically meaningful improvement while reducing false positives from normal measurement variation.

Step (h): Securing the Septal Electrode. The septal electrode is secured at the depth corresponding to confirmed left bundle branch capture, ensuring that the optimal position identified during ECGi mapping is maintained permanently. Once the optimal depth is confirmed by ECGi and the electrode is mechanically stable, the lead is secured and the procedure completed.

### ECGi System Technical Specifications

The electrocardiographic imaging (ECGi) system comprises several technical components:

Body Surface Electrode Array. A plurality of body surface electrodes numbering at least 64 electrodes, preferably 120 to 256 electrodes, distributed around the patient's torso. The electrodes are typically arranged in standardized spatial arrays, such as the vest-like configurations used in validated ECGi systems. The specific electrode positions have been established through prior clinical validation studies.

Cardiac Geometry Acquisition. A patient-specific three-dimensional cardiac geometry obtained from computed tomography or magnetic resonance imaging. High-quality imaging with sub-millimeter resolution for computed tomography or 1.5-3 millimeter resolution for magnetic resonance imaging provides the anatomic detail necessary for accurate electrical reconstruction. The geometry includes the epicardial and endocardial surfaces and is typically segmented from volumetric imaging data.

Computational Processing Module. Computational processing configured to reconstruct epicardial and/or endocardial electrical activation patterns by solving the inverse problem of electrocardiography. This inverse solution uses body surface potential measurements recorded from the electrode array and the cardiac geometry to estimate the electrical activation pattern on the ventricular epicardium and/or endocardium. Various mathematical algorithms can be employed for the inverse solution, including methods based on regularization, statistical approaches, or artificial intelligence-based methods.

Activation Map Generation. Real-time or near-real-time generation of ventricular activation maps displaying isochrones of electrical propagation. The maps show contours of constant activation time, creating a visual representation of the temporal sequence of depolarization. The maps use color coding to represent time, such as red for the earliest activation and blue for the latest activation.

Activation Time Measurement. Measurement of left ventricular activation time as the time elapsed between the earliest and latest activation points on the left ventricle. This represents the total duration of ventricular depolarization and is a standard measure of electrical resynchronization. The computation identifies the earliest and latest activated voxels within the left ventricle and calculates the time difference.

### Imageless ECGi Implementation

In a significant practical advancement, the real-time activation maps may be generated without requiring a pre-procedural imaging study, dramatically simplifying the clinical workflow:

Artificial Intelligence-Derived Geometric Models. An artificial intelligence-derived generic cardiac geometric model adapted to the patient's torso geometry is utilized. Machine learning algorithms trained on large populations of patient imaging data can create patient-specific cardiac geometry estimates based on simple anthropometric measurements (such as height, weight, and torso circumference), electrode array position determined from position tracking, and body surface potential measurements. These artificial intelligence models can generate a reasonable cardiac geometry estimate without requiring pre-procedural imaging.

Imageless Electrocardiographic Imaging Implementation. Imageless electrocardiographic imaging performs reconstruction of activation patterns based solely on body surface potential measurements and computational algorithms, without requiring pre-procedural computed tomography or magnetic resonance imaging. This approach dramatically simplifies the clinical workflow by enabling ECGi deployment in the electrophysiology laboratory without requiring advanced imaging studies, avoiding the delay of scheduling imaging, and eliminating radiation exposure or magnetic resonance imaging contraindications that might prevent imaging.

Rapid Visual Feedback to the Implanting Operator. Visual feedback to the implanting physician is provided within 5 seconds of each test pacing maneuver, or preferably within 2-5 seconds. This rapid feedback cycle enables real-time optimization of electrode position based on observed changes in activation patterns on successive activation maps. The operator can see results of each repositioning almost immediately.

Iterative Electrode Optimization Process. Electrode depth and position are adjusted based on observed changes in activation patterns on successive activation maps. The operator advances or withdraws the electrode by 1-2 millimeters and observes the corresponding change in ECGi-derived activation patterns on successive maps, iteratively optimizing position to maximize electrical resynchronization. The operator can observe the activation pattern change in real-time on the ECGi display, providing direct feedback regarding the effect of the repositioning.

### Advanced ECGi Capabilities and Measurements

The electrocardiographic imaging (ECGi) system may further provide:

Quantitative Electrical Resynchronization Measurement. Calculating total left ventricular activation time before and during pacing, providing objective quantification of the degree of electrical resynchronization achieved. Significant shortening of activation time of at least 15-20 percent indicates successful conduction system engagement. The system records baseline left ventricular activation time without pacing, then calculates left ventricular activation time at each septal pacing depth, allowing direct comparison.

Latest Activation Site Visualization. Visualization of the site of latest electrical activation in the left ventricle during baseline rhythm. This identifies the region of latest depolarization, which is typically the target for pacing therapy optimization. The ECGi map displays this site distinctly, usually as the most distal point of the latest color code.

Pacing Site Proximity to Left Bundle Branch Assessment. Assessment of proximity between the septal electrode pacing site and the left bundle branch by analyzing the pattern of early septal activation. Rapid septal activation that is consistent with capture near the specialized conduction tissue suggests proximity to the left bundle branch. The activation maps show the septal region activating very early (red color on the map), indicating the pacing site is near the conduction system rather than in remote myocardium.

Anodal Capture Detection. Detection of anodal capture from a bipolar pacing lead by identifying additional early right ventricular activation sites. In bipolar pacing, the anode (return electrode) may capture myocardium in addition to or instead of the intended cathode stimulation. If anodal capture is present, right ventricular sites would show early activation in addition to the intended septal activation. This is typically undesirable and the pacing parameters should be adjusted.

Electrical Resynchronization Assessment Reports. Generation of a report indicating whether electrical resynchronization has been achieved, defined as shortening of left ventricular activation time by at least 20 percent. These reports provide objective documentation of the quality of pacing therapy and may be stored in the patient's medical record for future reference and in-hospital decision documentation.

Alternative Noninvasive Mapping Modalities

The real-time verification methodology may be performed using alternative noninvasive electrocardiographic mapping techniques, providing options appropriate for different clinical settings and available equipment:

Body Surface Potential Mapping (BSPM). Body surface potential mapping provides an intermediate approach between standard 12-lead electrocardiography and full electrocardiographic imaging reconstruction. BSPM comprises:
Component 1: A dense array of body surface electrodes numbering at least 64 electrodes, preferably up to 252 electrodes, providing high spatial resolution of surface potentials.
Component 2: Recording of electrical potentials from the plurality of electrodes during cardiac pacing at each septal depth.
Component 3: Generation of potential maps or integral maps displaying spatial and temporal distribution of cardiac electrical activity on the body surface.
Component 4: Identification of activation timing at different regions of the left ventricle based on body surface potential patterns, using the relationship between surface potentials and underlying cardiac electrical activity. The temporal changes in the body surface potential distribution can be analyzed to infer the underlying ventricular activation sequence.

Body surface potential mapping offers intermediate complexity and cost compared to full ECGi while providing substantially more spatial detail than conventional 12-lead electrocardiography.

High-Resolution Multi-Lead Electrocardiography. This approach comprises:
Component 1: At least 12 leads for standard coverage (preferably 18-64 leads for enhanced spatial resolution), simultaneously recording electrocardiographic signals from multiple sites on the chest. Simultaneous recording from many leads provides comprehensive coverage of the chest.
Component 2: Simultaneous recording of electrocardiographic signals from multiple leads at fast sweep speeds of 100-250 millimeters per second, providing high temporal resolution. The higher sweep speed allows detection of small timing differences between leads.
Component 3: Analysis of regional activation times by measuring intervals from pacing stimulus to QRS changes at different leads. The time from pacing stimulus to the appearance of initial QRS deflection or the peak deflection at each lead is measured.
Component 4: Detection of small timing differences and morphologic changes indicative of the direction of electrical impulse propagation. Earlier QRS changes at inferior leads versus superior leads would suggest apex-to-base propagation.

While less sophisticated than full ECGi, high-resolution multi-lead electrocardiography may be deployed in standard electrophysiology laboratories without requiring computed tomography or magnetic resonance imaging, or extensive computational analysis.

### Application to Activation Direction and Timing Assessment

These and other similar and suitable noninvasive electrocardiographic mapping techniques are used to determine the direction of electrical impulse propagation along the lateral wall of the left ventricle and to measure the time delay from electrical activation of the interventricular septum to electrical activation of the lateral wall. The noninvasive measurements may supplement or replace intracardiac electrode measurements in determining whether left bundle branch capture has been achieved. Additionally, these noninvasive measurements may be used for chronic monitoring to determine whether sustained capture is maintained. For example, a patient could undergo high-resolution multi-lead electrocardiography at each device check-in to assess activation patterns without requiring lead-based electrograms.

### Clinical Examples Demonstrating Method Implementation

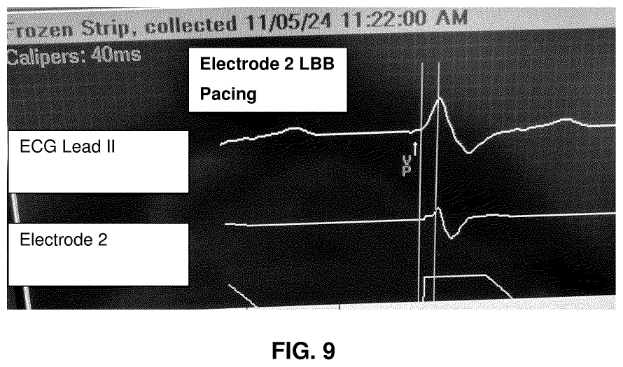

### Example 1: Successful Left Bundle Branch Capture with Multimodal Verification, FIGS. 2-9

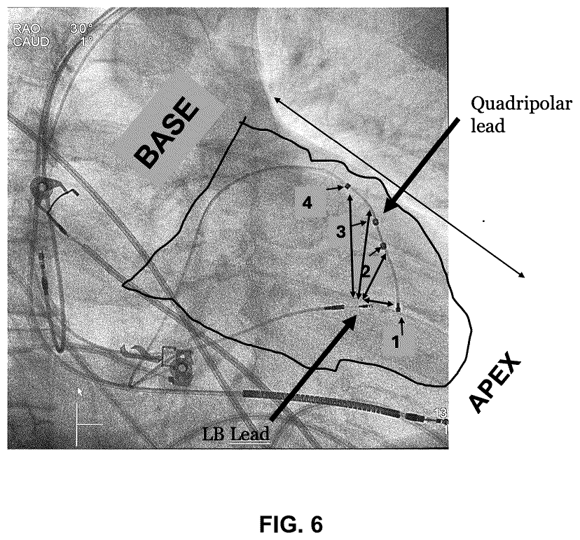

A 68-year-old patient with left bundle branch block, nonischemic cardiomyopathy and heart failure with reduced ejection fraction (HFrEF, ejection fraction 30%) presented for septal electrode implantation to achieve left bundle branch area pacing (LBBAP). During the procedure, a quadripolar coronary sinus lead (Quadripolar lead, arrow) with electrodes 1-4 (small arrows) positioned apex-to-base (arrow) and a septal electrode (LB lead, arrow) was deployed, as shown in the fluoroscopic image of the heart in RAO view in FIG. 6. The patient had a baseline QRS duration of 150 milliseconds, clearly demonstrating left bundle branch block.

Baseline Measurements (pre-pacing Assessment):

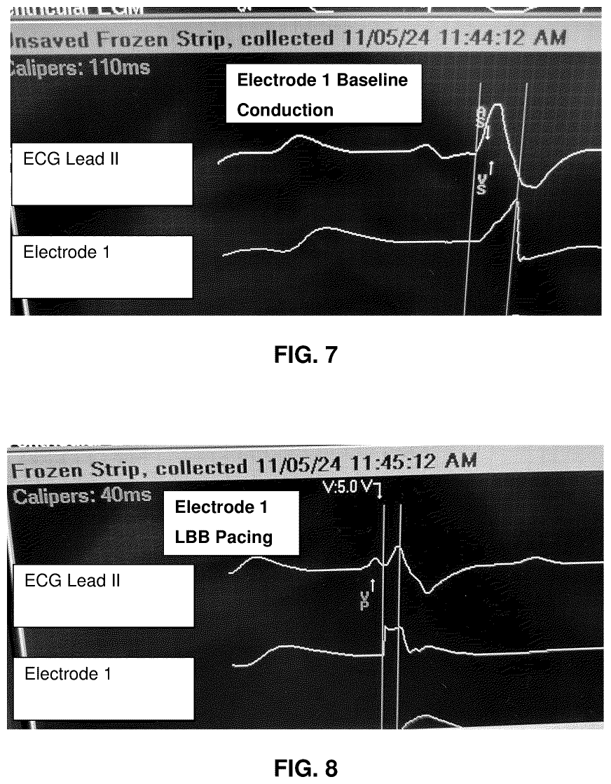

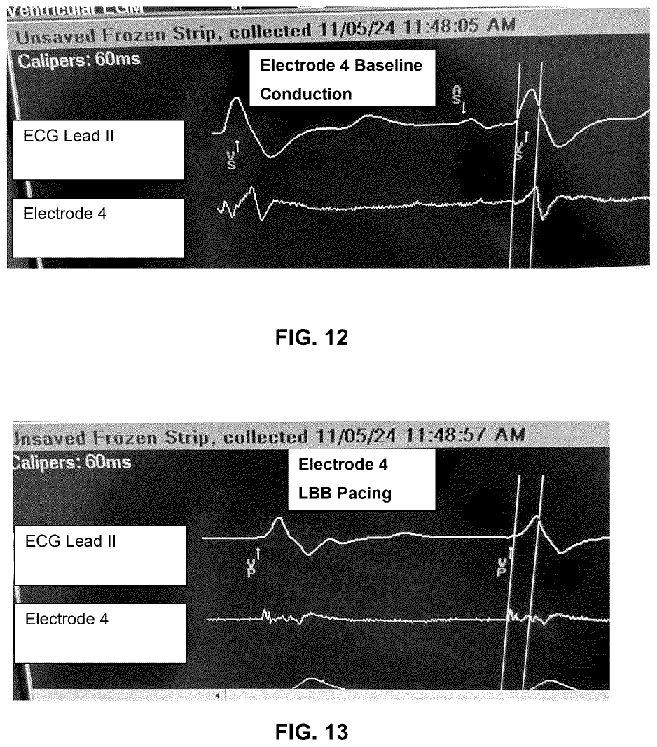

Direction of electrical impulse propagation: Base-to-apex, representing abnormal conduction consistent with LBBB. Activation times from QRS onset to peak local lateral wall electrogram measurements were as follows: Electrode 1 (apical position) equals 110 milliseconds (FIG. 7-13 show unipolar timing measurements at individual electrodes 1-4 reflecting either very slow native conduction during LBBB-labeled, or LBB pacing labeled), Electrode 2 equals 100 milliseconds, Electrode 3 equals 80 milliseconds, and Electrode 4 (basal position) equals 60 milliseconds (see individual figures). This sequence showed the earliest activation at Electrode 4 (basal, FIG. 7) and the latest activation at Electrode 1 (apical, FIG. 12), confirming the base-to-apex propagation direction.

This pattern is consistent with LBBB, where impulses reach the basal region from the right bundle branch and then propagate slowly across myocardium toward the apex.

In this patient, LBBB was present with documented conduction system disease and proven failure of normal left bundle branch propagation.

### Upon Initiating Septal Pacing:

Direction of electrical impulse propagation: Reversed to apex-to-base, representing the physiological normal direction, see FIGS. 8, 9, 11, and 13. This reversal from an abnormal baseline represents a reversal of propagation direction and meets the criterion for successful left bundle branch capture.

Activation delays from the pacing stimulus to the peak local lateral wall electrogram: Electrode 1 equals 40 milliseconds, Electrode 2 equals 40 milliseconds, Electrode 3 equals 40 milliseconds, and Electrode 4 equals 60 milliseconds, see FIGS. 8, 9, 11, and 13. This sequence showed rapid activation at electrodes 1-3 with minimal variation and delayed activation at Electrode 4. This pattern is characteristic of apex-to-base Purkinje propagation, where rapid Purkinje fibers reach the entire lateral wall almost simultaneously.

The minimal variation among electrodes 1-3 (less than 15 milliseconds spread) indicates synchronous lateral wall activation characteristic of Purkinje conduction, in contrast to myocardial conduction, which would show progressive delay.

Total left ventricular activation time shortened from approximately 110 milliseconds (baseline) to approximately 60 milliseconds (with pacing), representing a 45 percent improvement, substantially exceeding the 15 percent threshold.

Quantitative criteria: Time delay from pacing stimulus to lateral wall activation equals 40 milliseconds, well below the 80 millisecond threshold for LBB capture, and substantially shorter than the baseline 110 millisecond delay. This 40 millisecond delay is consistent with rapid Purkinje conduction velocities of 2-3 meters per second across the septal-to-lateral wall distance.

### Bidirectional Pacing Measurements (Functional Confirmation):

Anterograde measurement (septal to lateral wall): Mean conduction time from septal electrode to lateral wall electrodes equals 45 milliseconds. This rapid conduction time is characteristic of Purkinje fiber engagement.

Retrograde measurement (lateral wall to septal): When pacing from Electrode 2, conduction time to septal electrode equaled 95 milliseconds (not shown). This prolonged retrograde time reflects slow myocardial conduction unable to effectively utilize the conduction system in the retrograde direction.

### Diagnostic Conclusion:

The convergence of multiple independent diagnostic criteria all confirmed successful left bundle branch capture: (1) reversal of propagation direction from abnormal baseline base-to-apex to physiologic apex-to-base (Criterion 1 met); (2) substantial shortening of activation delays from 110 milliseconds to 40 milliseconds, representing 64 percent improvement, far exceeding the 15 percent threshold (Criterion 2 met); (3) minimal variation in lateral wall electrode activation times (less than 15 milliseconds) indicating synchronous Purkinje activation (Criterion 3 met); (4) bidirectional conduction time differential of 50 milliseconds substantially exceeding the patient-specific threshold of 30 milliseconds (Bidirectional criterion met); and (5) time delay of 40 milliseconds well below the 80 millisecond threshold for LBB capture (Quantitative timing criterion met).

All diagnostic approaches provided concordant evidence of successful capture. The septal electrode was secured at this position.

### Clinical Outcome:

Within 3 months, the patient demonstrated left ventricular ejection fraction improvement from 30 percent to 45 percent, representing a substantial 15 percentage point absolute improvement in ejection fraction. Electrocardiographic QRS duration reduced from 150 milliseconds to 105 milliseconds, representing a 30 percent narrowing. The patient demonstrated symptomatic improvement from New York Heart Association Class III dyspnea on exertion and activity limitation to New York Heart Association Class II with improved exercise tolerance and reduced dyspnea. These substantial clinical improvements confirmed that successful left bundle branch capture was maintained, providing therapeutic benefit.

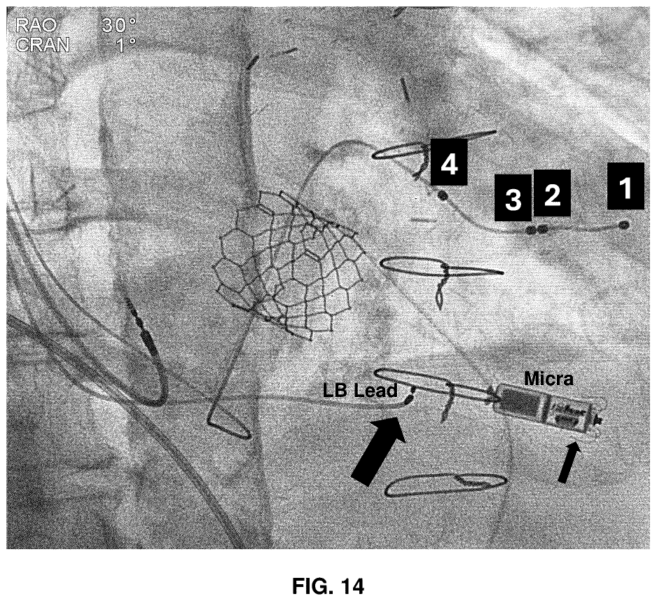

### Example 2: Septal Myocardial Capture Without Left Bundle Branch Engagement, FIGS. 14-16

A 72-year-old patient with a Micra leadless pacemaker positioned on the right side of the interventricular septum presented with right ventricular pacing-induced cardiomyopathy. The patient had become pacemaker-dependent due to complete heart block and received 100 percent ventricular pacing from the right ventricular septal location. The chronic right ventricular pacing resulted in ventricular dyssynchrony very similar to left bundle branch block, causing heart failure development with ejection fraction decline to 32 percent.

The patient was referred for septal electrode implantation to transition from right ventricular pacing to left bundle branch pacing to correct the pacing-induced cardiomyopathy. A quadripolar coronary sinus lead was placed for sensing and biventricular pacing capability. A septal electrode was advanced into the interventricular septum. FIG. 14 shows CXR in RAO view demonstrating Micra leadless pacemaker—small arrow, quadripolar coronary sinus lead with electrodes 1,2,3,4 numbered and septal lead large arrow. This patient had a baseline paced QRS duration of 160 milliseconds from the existing right ventricular pacing, representing substantial dyssynchrony.

Baseline Measurements (pre-pacing Assessment With New Septal Electrode):

Direction of electrical impulse propagation: The baseline pattern was complex (FIG. 15 shows timing measurements at baseline-RV Pace Micra and during LV septal pacing at high and low output). The existing Micra leadless pacemaker on the right ventricular septum was depolarizing the right side of the heart and resulting in very delayed activation at the lateral LV wall (electrodes 1-4, delay 140-137-144-146 milliseconds). However, the new septal electrode had not yet been activated. The baseline pattern showed predominant right-to-left propagation with characteristics of right ventricular pacing. The direction was not clearly apex-to-base or base-to-apex but showed characteristics of lateral activation occurring with very significant delay, resulting in dyssynchrony and heart failure.

Activation delays from QRS onset: All four electrodes (1, 2, 3, and 4) showed approximately equal activation times of around 140 milliseconds. This uniform delay without clear sequence indicated globally delayed and dyssynchronous left ventricular activation. The small variations among electrode times indicated lack of clear directional propagation, consistent with slow, dyssynchronous myocardial conduction reaching all sites at nearly the same time.

### Upon Initiating Pacing From New Septal Electrode:

Direction of electrical impulse propagation: The direction remained predominantly base-to-apex or showed mixed direction without a clear apex-to-base pattern irrespective of pacing output (FIG. 15, lines 2 and 3). The activation pattern did not reverse to the physiologic apex-to-base sequence that would indicate LBB capture.

Activation delays from pacing stimulus: Electrode 1 equals 125 milliseconds, Electrode 2 equals 117 milliseconds, Electrode 3 equals 133 milliseconds, and Electrode 4 equals 133 milliseconds. These delays showed only minimal change compared to the baseline delay of 140 milliseconds. The changes were all within the 15 percent threshold for significant change: 125/140 equals 89 percent of baseline (11 percent decrease, below 15 percent threshold), 117/140 equals 84 percent (16 percent decrease, just above threshold), 133/140 equals 95 percent (5 percent decrease, below threshold), and 133/140 equals 95 percent (5 percent decrease, below threshold).

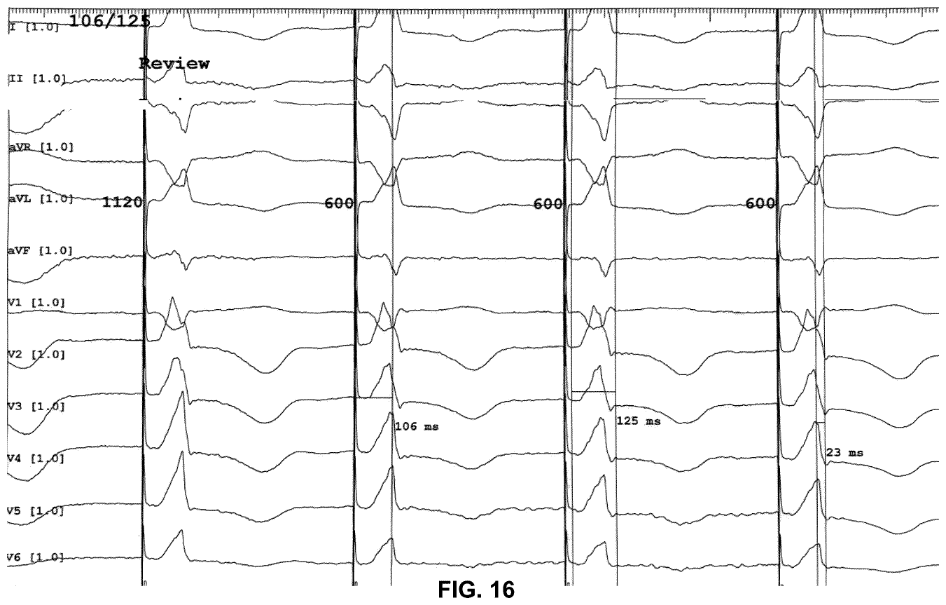

Overall, the activation delays did not substantially shorten irrespective of pacing output, indicating that conduction system engagement was not achieved. The pattern did not indicate restoration of rapid Purkinje conduction. The septal electrode could not be advanced any deeper into the septum and left bundle branch capture was not achieved. Equal delay at high and low pacing output also indicated significant distance to conduction system and inability to engage it even with higher pacing voltage. Paced 12-lead ECG (FIG. 16) showed some features consistent with conduction system capture (relatively narrow QRS, isoelectric line between the stimulus and QRS onset, R′ in lead V1) in the setting of underlying conduction system disease, while timing measurements (R wave peak time and similar were more consistent with septal capture). Quadripolar lead measurements (FIG. 15) confirmed that and decision was made to proceed with biventricular pacing.

### Diagnostic Conclusion:

The objective physiologic measurements clearly indicated septal myocardial capture without left bundle branch engagement, despite the ambiguous standard electrocardiographic appearance. All diagnostic criteria converged on this conclusion:

Direction criterion: Absence of reversal to apex-to-base propagation (Direction criterion NOT met).

Timing criterion: Failure to shorten activation delays by more than 15 percent (Timing criterion NOT met). Changes were within or barely exceeding 15 percent threshold, not the robust shortening seen with true capture.

None of the diagnostic criteria indicated successful LBB capture. Objective measurements from the bidirectional pacing protocol proved critical in this case because standard electrocardiography was ambiguous and could have led to acceptance of inadequate capture.

### Clinical Decision and Therapy Adjustment:

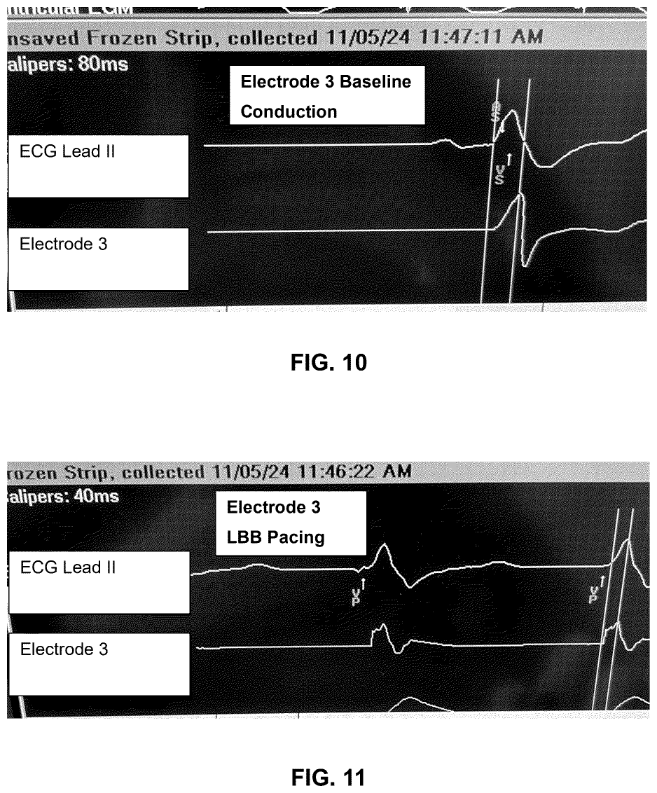

Based on the objective findings from the bidirectional pacing method, the clinical team made an informed decision to switch to biventricular heart pacing rather than accepting septal myocardial capture (left bundle branch capture could not be achieved despite multiple attempts). Biventricular pacing was programmed, utilizing the new septal electrode and the quadripolar coronary sinus lead already implanted, see FIG. 10. Optimal atrioventricular delay (typically 100-150 milliseconds) and interventricular delay (typically 0-40 milliseconds) were programmed. The interventricular delay was adjusted to optimize the timing of depolarization between the septal and lateral wall sites.

### Clinical Outcome:

Within 6 weeks, the patient demonstrated left ventricular ejection fraction improvement from 32 percent to 42 percent, a 10 percentage point improvement. The improvement with biventricular pacing was substantial but somewhat less robust than typically seen with true LBB capture in other patients. Symptomatic improvement was noted with ejection fraction improvement, though the patient did not achieve as complete a recovery as Example 1. This residual limitation is consistent with biventricular pacing being somewhat less physiologic than true LBB capture.

This example illustrates the following. The objective measurements prevented acceptance of inadequate myocardial capture and guided implementation of an alternative strategy that, while providing some benefit, allowed accurate prognostic counseling regarding expected outcomes. The case demonstrates how objective physiologic measurements can resolve diagnostic uncertainty and guide optimal clinical decision-making.

### Integration of Multiple Diagnostic Approaches

### Comprehensive Multimodal Assessment Strategy

A patient undergoing septal electrode implantation for left bundle branch pacing may be evaluated using integrated multiple diagnostic approaches to maximize diagnostic accuracy and provide complementary information:

Real-Time ECGi-Guided Mapping During Implantation. Electrocardiographic imaging provides visual feedback of activation patterns as the electrode is advanced to different depths within the septum. The operator observes the transition from septal myocardial pacing patterns (delayed lateral wall activation with abnormal sequences) to left bundle branch pacing patterns (early septal activation with rapid, synchronous lateral wall activation in apex-to-base direction). The ECGi maps guide the operator to the depth at which the activation pattern transitions to the desired left bundle branch pattern and at which left ventricular activation time is optimized.

Simultaneous Bidirectional Pacing Measurements. At the optimal ECGi-identified depth, bidirectional pacing measurements may be performed, confirming that the electrical pattern identified visually on ECGi maps corresponds to true conduction system engagement. The large bidirectional delta (exceeding patient-specific threshold) provides quantitative functional confirmation that the activation pattern seen on ECGi reflects Purkinje system capture rather than just faster myocardial conduction.

Quantitative Timing Measurements. Direct measurements of time delays from septal electrode to lateral wall electrodes confirm the rapid conduction times expected from Purkinje fiber engagement (typically 40-70 milliseconds rather than 80-100 milliseconds for myocardium).

Activation Direction Measurement. Assessment confirms restoration of physiologic apex-to-base propagation by comparing arrival times at apical versus basal electrodes.

Standard Electrocardiography. A 12-lead electrocardiogram is obtained to identify QRS narrowing and timing/morphologic features consistent with LBB capture. The electrocardiogram provides supportive evidence and allows comparison to baseline and to standard criteria used in prior published studies.

Integration Summary. This multimodal approach provides redundant confirmation of successful capture from multiple independent physiologic approaches. Different methods assess different aspects of the electrical activity (visual activation maps, temporal conduction properties, directional asymmetry, temporal delays, morphology). Convergence of all methods on the conclusion of successful capture provides high diagnostic confidence. Divergence of methods (for example, ECGi showing improved activation pattern but bidirectional delta below threshold) would prompt further evaluation, potential electrode repositioning, or decision to employ biventricular pacing.

### Clinical Implementation Considerations

This comprehensive assessment approach requires:

Trained operator expertise in interpreting ECGi maps and understanding the physiologic basis for bidirectional measurement.

Availability of ECGi system in the electrophysiology laboratory, either through institutional equipment or through vendor-provided mobile ECGi systems.

Additional time during the implantation procedure for ECGi setup and measurements, typically adding 15-30 minutes to procedure duration.

Cost of ECGi system deployment.

However, the improved diagnostic accuracy and reduced risk of accepting inadequate septal myocardial capture may justify these additional resources by avoiding late clinical failure of inadequate pacing therapy.

### Chronic Monitoring Integration and Long-term Surveillance

### Comprehensive Follow-up Surveillance Protocol

Following successful implantation confirmed by multimodal assessment, chronic monitoring maintains surveillance for loss of capture. The surveillance strategy involves:

Scheduled Device Check-Ins. Device interrogations typically occur every 3-12 months or more frequently if indicated. During device check-ins, the processing module automatically measures bidirectional conduction time delta, comparing the current delta to the patient-specific threshold established at implantation. A report is generated indicating whether the current delta remains suprathereshold, indicating continued conduction system engagement, or whether the delta has decreased below threshold, indicating loss of capture. The patient-specific threshold value established at implantation remains programmed in the device and is used for ongoing comparisons.

Remote Monitoring Capability. Implanted devices increasingly include remote monitoring capability, allowing periodic automatic transmission of device measurements including pacing thresholds, impedances, and electrogram recordings to a monitoring center. These transmitted measurements may include bidirectional conduction time delta measurements, with automatic alert generation if delta falls below threshold.

Activation Direction and Timing Assessment. During device check-ins, the device measures activation direction and time delays similar to implantation assessment. Any change in activation direction from apex-to-base baseline to abnormal patterns or any increase in time delays by more than 20 percent compared to implantation values would trigger alerts.

Automatic Corrective Actions. Modern implanted devices can implement automatic corrective actions upon detection of loss of capture. These may include increased pacing output to compensate for increased capture threshold, switch to unipolar pacing or biventricular pacing, or notifications to the clinician and patient triggering urgent evaluation.

Patient Symptom Reporting. Patients are educated to report symptoms that might indicate pacing failure, such as return of dyspnea, decreased exercise tolerance, or syncope. These symptoms may occur if left bundle branch capture is lost and myocardial pacing is inadequate for hemodynamic support.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method for detecting a capture of a left bundle branch of a heart for pacing purposes, the method comprising the following steps:
(a) deploying a septal electrode configured for left bundle branch pacing in an interventricular septum of a heart at a location intended for left bundle branch pacing,
(b) monitoring a direction of electrical impulse propagation along a lateral wall during left ventricle activation and measuring a time delay from an electrical activation of the interventricular septum to an electrical activation of the lateral wall,
(c) initiating cardiac pacing using the septal electrode of step (a),
(d) for patients with abnormal direction of electrical impulse propagation along the lateral wall of the left ventricle at baseline, identifying left bundle branch capture upon detecting: a restoration or change toward a physiologically normal apex-to-base direction of electrical impulse propagation along the lateral wall after initiation of cardiac pacing in step (c), OR a shortening of the time delay from electrical activation of the interventricular septum to electrical activation of the lateral wall by at least 15% compared to baseline, thereby indicating restoration of rapid conduction via the specialized conduction system,
(e) for patients with abnormal direction of electrical impulse propagation along the lateral wall at baseline, identifying left ventricular septal capture (absent left bundle branch capture) upon detecting: persistence or worsening of abnormal electrical impulse propagation direction after initiation of cardiac pacing in step (c), OR persistence of prolonged time delay from electrical activation of the interventricular septum to electrical activation of the lateral wall, wherein the time delay remains within 15% of the baseline delay or increases, thereby indicating absence of rapid conduction via the specialized conduction system and/or loss of normal apex-to-base activation pattern.

2. The method of claim 1, wherein for patients with normal baseline direction of electrical impulse propagation along the lateral wall of the left ventricle from apex-to-base, the method further comprises detecting preservation of the direction of electrical impulse propagation and detecting an absence of an increase of a delay between activation of the interventricular septum and the lateral wall by more than 10% of a baseline delay.

3. The method of claim 1, wherein step (b) is performed using at least two lateral wall electrodes configured to detect activation of a lateral wall of a left ventricle of the heart.

4. The method of claim 3, wherein step (d) is performed by comparing electrical impulse arrival times and respective locations of the at least two lateral wall electrodes spaced along the lateral wall of the left ventricle.

5. The method of claim 3, wherein step (b) is performed using a quadripolar lateral wall electrode placed in a coronary sinus along the lateral wall of the left ventricle of the heart.

6. The method of claim 1, wherein the time delay is measured as an interval from:
(i) the pacing stimulus delivered by the septal electrode to the peak of the R-wave in a selected lead on an electrocardiogram, OR
(ii) the pacing stimulus delivered by the septal electrode to the earliest detected electrical activation at at least two lateral wall electrodes, OR
(iii) the pacing stimulus delivered by the septal electrode to the peak electrical activation signal detected at at least two lateral wall electrodes, OR
(iv) the onset of the QRS complex on an electrocardiogram to the earliest detected electrical activation at at least two lateral wall electrodes.

7. The method of claim 1, further comprising identifying left bundle branch capture by detecting at least one of the following quantitative criteria:
shortening of the time delay of step (b) by at least 25%, indicating rapid conduction via the specialized conduction system, OR a time delay from the pacing stimulus to left ventricular lateral wall activation of less than 80 milliseconds, preferably less than 75 milliseconds, most preferably less than 70 milliseconds.

8. The method of claim 1, further comprising identifying left ventricular septal capture by detecting at least one of the following quantitative criteria:
the direction of electrical impulse propagation along the lateral wall continuing to be abnormal, including but not limited to base-to-apex, side-to-side, or other non-physiological patterns, OR a time delay from the pacing stimulus to left ventricular lateral wall activation exceeding 85 milliseconds, preferably exceeding 90 milliseconds, most preferably exceeding 95 milliseconds, indicating slow myocardial conduction without engagement of the specialized conduction system.

9. A method for differentiating between a left ventricular septal capture and a left bundle branch capture during heart pacing, the method comprising:
(a) deploying a septal electrode configured for left bundle branch pacing in an interventricular septum of a heart at a location intended for left bundle branch pacing,
(b) deploying at least two lateral wall electrodes along a lateral wall of a left ventricle, wherein the at least two lateral wall electrodes are spaced apart along a direction between an apex and a base of the heart,
(c) initiating a first pacing protocol by pacing from the septal electrode and measuring a first conduction time from a pacing stimulus delivered by the septal electrode to electrical activation detected at the at least two lateral wall electrodes,
(d) initiating a second pacing protocol by pacing from at least one of the at least two lateral wall electrodes and measuring a second conduction time from a pacing stimulus delivered by said at least one lateral wall electrode to electrical activation detected at the septal electrode,
(e) calculating a bidirectional conduction time differential (delta) as an absolute difference between the first conduction time and the second conduction time,
(f) identifying left bundle branch capture when the bidirectional conduction time differential exceeds a threshold value, wherein: the first conduction time is shorter than the second conduction time, indicating rapid anterograde conduction through the specialized left bundle branch conduction system when pacing from the septal electrode, and slower retrograde myocardial conduction when pacing from the lateral wall electrodes, and the threshold value is selected from the range of 10 milliseconds to 50 milliseconds, preferably 15 milliseconds to 35 milliseconds, most preferably 20milliseconds to 30 milliseconds,
(g) identifying left ventricular septal capture when the bidirectional conduction time differential is less than the threshold value, indicating comparable slow myocardial conduction in both pacing directions and absence of specialized conduction system engagement.

10. The method of claim 9, wherein:
(a) the at least two lateral wall electrodes comprise a quadripolar electrode lead with four electrodes numbered 1 through 4, positioned with electrode 1 closest to the apex and electrode 4 closest to the base,
(b) step (c) further comprises measuring the first conduction time from the septal electrode to each of electrodes 1, 2, 3, and 4 individually,
(c) step (d) further comprises pacing sequentially from each of electrodes 1, 2, 3, and 4 individually and measuring the second conduction time from each respective electrode to the septal electrode,
(d) step (f) further comprises identifying left bundle branch capture when: the bidirectional conduction time differential exceeds the threshold value for at least two of the four electrodes, AND the first conduction time measured from the septal electrode to electrodes 1, 2, 3, and 4 shows minimal variation (less than 15 milliseconds difference between earliest and latest activation among the four electrodes), indicating rapid and synchronous activation of the lateral wall via the specialized conduction system, whereas the second conduction time measured when pacing from any of electrodes 1, 2, 3, or 4 to the septal electrode shows longer delays exceeding the first conduction time by at least the threshold value, indicating slow myocardial conduction unable to effectively engage the specialized conduction system in the retrograde direction due to unidirectional conduction properties of Purkinje fibers.

11. The method of claim 9, wherein the threshold value for the bidirectional conduction time differential is dynamically adjusted based on one or more of the following patient-specific factors:
(a) baseline QRS duration in intrinsic rhythm,
(b) presence or absence of left bundle branch block,
(c) presence or absence of ischemic heart disease or myocardial scarring,
(d) left ventricular ejection fraction,
wherein patients with left bundle branch block or wider baseline QRS duration utilize a higher threshold value of 25 to 40 milliseconds, and patients with narrow baseline QRS utilize a lower threshold value of 10 to 24 milliseconds, or of normal conduction delay of 10 milliseconds or more.

12. The method of claim 9, further comprising:
(a) performing the bidirectional pacing measurements at multiple pacing output voltages ranging from threshold capture voltage to a voltage at least 2-fold higher than the threshold capture voltage,
(b) confirming left bundle branch capture when the bidirectional conduction time differential remains consistently above the threshold value across multiple pacing output voltages, indicating stable capture of the specialized conduction system,
(c) identifying loss of left bundle branch capture when the bidirectional conduction time differential decreases below the threshold value at lower pacing outputs, indicating loss of conduction system engagement and transition to septal myocardial capture only.

13. The method of delivering a pacing therapy to a heart, comprising the following steps:
(a) deploying a septal electrode configured for left bundle branch pacing in an interventricular septum of a heart at a location intended for left bundle branch pacing,
(b) deploying at least two lateral wall electrodes along a lateral wall of a left ventricle,
(c) detecting left bundle branch capture by (i) initiating heart pacing using the septal electrode and (ii) using the bidirectional pacing method of claim 9 to confirm left bundle branch capture,
(d) if the left bundle branch is captured as confirmed in step (c), continuing to deliver the pacing therapy to the heart using the septal electrode,
(e) if the left bundle branch cannot be captured as confirmed in step (c), initiating biventricular heart pacing using at least the septal electrode and at least one lateral wall electrode.

14. The method of monitoring a left bundle branch capture during chronic heart pacing using a septal electrode positioned in an interventricular septum, the method comprising:
(a) performing periodic or continuous monitoring of at least one of: a direction of electrical impulse propagation along a lateral wall of a left ventricle using implanted lateral wall electrodes or noninvasive electrocardiographic mapping, a time delay from electrical activation of the interventricular septum to electrical activation of the lateral wall, a bidirectional conduction time differential measured by alternating pacing from the septal electrode and from lateral wall electrodes using the method of claim 9,
(b) detecting loss of left bundle branch capture upon identifying at least one of: a change in the direction of electrical impulse propagation from apex-to-base to a non-apex-to-base pattern, an increase in the time delay by more than 20% compared to a baseline measurement obtained when left bundle branch capture was confirmed, a decrease in the bidirectional conduction time differential to below the threshold value, indicating loss of specialized conduction system engagement,
(c) upon detecting loss of left bundle branch capture, initiating at least one corrective action selected from: alerting a patient or healthcare practitioner, adjusting pacing parameters including output voltage, pulse width, polarity, or pacing site, automatically switching to biventricular pacing using the septal electrode and at least one lateral wall electrode, scheduling a follow-up evaluation or device interrogation.

15. A system for detecting left bundle branch capture comprising:
(a) a septal electrode configured for deployment in an interventricular septum and delivery of pacing stimuli,
(b) at least two lateral wall electrodes configured for deployment along a lateral wall of a left ventricle and spaced apart along a direction between an apex and a base of the heart, wherein the at least two lateral wall electrodes are configured to sense electrical activation of the lateral wall,
(c) a pacing module configured to: deliver pacing stimuli from the septal electrode at adjustable pacing outputs, deliver pacing stimuli from the at least two lateral wall electrodes,
(d) a sensing module configured to: receive electrical activation signals from the at least two lateral wall electrodes when pacing from the septal electrode, receive electrical activation signals from the septal electrode when pacing from the at least two lateral wall electrodes,
(e) a processing module configured to: determine a sequence of electrical activation among the at least two lateral wall electrodes to identify a direction of electrical impulse propagation, measure a first conduction time from pacing stimuli delivered by the septal electrode to electrical activation detected at the at least two lateral wall electrodes, measure a second conduction time from pacing stimuli delivered by the at least two lateral wall electrodes to electrical activation detected at the septal electrode, calculate a bidirectional conduction time differential as an absolute difference between the first conduction time and the second conduction time, compare the bidirectional conduction time differential to a threshold value,
(f) an output module configured to: indicate left bundle branch capture when the bidirectional conduction time differential exceeds the threshold value and the direction of electrical impulse propagation is apex-to-base, indicate left ventricular septal capture when the bidirectional conduction time differential is less than the threshold value or the direction of electrical impulse propagation is non-apex-to-base.

16. A method of real-time verification of left bundle branch capture during septal electrode implantation, comprising:
(a) deploying an electrocardiographic imaging (ECGi) system comprising a plurality of body surface electrodes prior to or during the implantation procedure,
(b) acquiring a three-dimensional cardiac geometry from computed tomography or magnetic resonance imaging, or using an artificial intelligence-derived geometric model,
(c) continuously recording electrical signals from the plurality of body surface electrodes during the implantation procedure,
(d) generating real-time or near-real-time activation maps showing ventricular electrical activation patterns as the septal electrode is advanced into the interventricular septum,
(e) initiating test pacing from the septal electrode at multiple depths of septal penetration and generating corresponding activation maps for each pacing maneuver,
(f) comparing the activation maps obtained at different septal depths to identify a transition from: a left ventricular septal pacing pattern characterized by lateral left ventricular activation occurring with delayed timing and abnormal activation sequence, to a left bundle branch pacing pattern characterized by early septal activation followed by rapid and synchronous left ventricular lateral wall activation with restoration of physiological apex-to-base activation and shortened left ventricular activation time,
(g) confirming successful left bundle branch capture when the activation map demonstrates the left bundle branch pacing pattern and left ventricular activation time is shortened by at least 15% compared to baseline or compared to more superficial septal pacing sites,
(h) securing the septal electrode at the depth corresponding to confirmed left bundle branch capture.

17. The method of claim 16, wherein the electrocardiographic imaging (ECGi) system comprises:
(a) a plurality of body surface electrodes numbering at least 64 electrodes, distributed around the patient's torso,
(b) a patient-specific three-dimensional cardiac geometry obtained from computed tomography or magnetic resonance imaging,
(c) computational processing configured to reconstruct epicardial and/or endocardial electrical activation patterns,
(d) real-time or near-real-time generation of ventricular activation maps displaying isochrones of electrical propagation,
(e) measurement of left ventricular activation time as a time elapsed between an earliest and a latest activation points on the left ventricle.

18. The method of claim 16, wherein the real-time activation maps in step (d) are generated without requiring a pre-procedural imaging study by:
(a) utilizing an artificial intelligence-derived generic cardiac geometric model adapted to the patient's torso geometry,
(b) performing imageless electrocardiographic imaging that reconstructs activation patterns based solely on body surface potential measurements and computational algorithms,
(c) providing visual feedback to the implanting physician within 5 seconds of each test pacing maneuver,
(d) enabling adjustment of septal electrode depth and position based on observed changes in activation patterns on successive activation maps.

19. The method of claim 16, wherein the electrocardiographic imaging (ECGi) system further provides:
(a) quantitative measurement of electrical resynchronization by calculating total left ventricular activation time before and during pacing,
(b) visualization of the site of latest electrical activation in the left ventricle during baseline rhythm,
(c) assessment of proximity between the septal electrode pacing site and the left bundle branch by analyzing the pattern of early septal activation,
(d) detection of anodal capture from a bipolar pacing lead by identifying additional early right ventricular activation sites,
(e) generation of a report indicating whether electrical resynchronization has been achieved, defined as shortening of left ventricular activation time by at least 20%.

20. The method of claim 16, wherein step (d) is performed using noninvasive electrocardiographic mapping comprising one or more of the following:
(a) electrocardiographic imaging (ECGi) as defined in claim 17,
(b) body surface potential mapping (BSPM) comprising: a dense array of body surface electrodes numbering at least 64 electrodes, recording of electrical potentials from the plurality of electrodes during cardiac pacing, generation of potential maps or integral maps displaying spatial and temporal distribution of cardiac electrical activity, identification of activation timing at different regions of the left ventricle based on body surface potential patterns,
(c) high-resolution multi-lead electrocardiography comprising at least 12 leads, simultaneous recording of electrocardiographic signals from multiple leads, analysis of regional activation times, and detection of small timing differences indicative of the direction of electrical impulse propagation,
wherein the noninvasive electrocardiographic mapping is used to determine the direction of electrical impulse propagation along the lateral wall of the left ventricle and to measure the time delay from electrical activation of the interventricular septum to electrical activation of the lateral wall.
